(19) United States
(12) Patent Application Publication
(10) Pub. No.: US 2014/0336259 A1
(43) Pub. Date: Nov. 13, 2014

(54) PHENOLIC COMPOUNDS WITH ANTIOXIDANT AND ANTI-CANCER PROPERTIES, ANALOGS AND SYNTHESIS THEREOF

(71) Applicants: Navindra Seeram, Charlestown, RI (US); Julie Barbeau, Boucherville (CA); Genevieve BeLand, Saint-Hyacinthe (CA); Keykavous Parang, Saunderstown, RI (US)

(72) Inventors: Navindra Seeram, Charlestown, RI (US); Julie Barbeau, Boucherville (CA); Genevieve BeLand, Saint-Hyacinthe (CA); Keykavous Parang, Saunderstown, RI (US)

(21) Appl. No.: 14/125,237

(22) PCT Filed: Jun. 11, 2012
(86) PCT No.: PCT/CA2012/000556

§ 371 Date: Jul. 22, 2014

Related U.S. Application Data
(60) Provisional application No. 61/495,574, filed on Jun. 10, 2011.

(51) Int. Cl. C07C 67/343; C07C 39/15; C07C 41/30; C07C 41/22; C07C 41/26; C07C 59/68; C07C 43/205

(52) U.S. Cl. CPC C07C 67/343; C07C 59/68; C07C 39/15; C07C 43/2055; C07C 41/22; C07C 41/26; C07C 41/30
USPC 514/570; 514/568; 514/721; 514/734; 560/57; 568/640; 568/641

(57) ABSTRACT

The present document describes a phytochemical isolated from maple syrup and composition comprising the same. More specifically, the document describes an antioxidant phytochemical compound, derivates thereof, and composition comprising the same. The document also describes a process of synthesizing the antioxidant phytochemical compound.

### CROSS-REFERENCE TO RELATED APPLICATIONS

This application claims priority of U.S. provisional patent application 61/495,574 filed on Jun. 10, 2011.

### BACKGROUND

(a) Field

The subject matter disclosed generally relates to a phytochemical isolated from maple syrup and composition comprising the same. More specifically, the subject matter relates to an antioxidant phytochemical compound, derivates thereof, and composition comprising the same. The subject matter also relates to a process of synthesizing the antioxidant phytochemical compound.

(b) Related Prior Art

Maple syrup (MS) is a natural product obtained by thermal evaporation of sap collected from certain maple (Acer) species including the sugar maple (A. saccharum) tree. The province of Quebec in Canada is the largest producer of MS, and this premium natural sweetener is popularly consumed worldwide. Thus, identification of the chemical constituents, beyond the natural sugars (sucrose), of MS is of great interest from a human health perspective. MS contains a diverse range of phenolic compounds which are naturally present in the xylem sap and concentrated in syrup. The identification of these new phenolic compounds may lead to the foundation of a new class of compounds with health beneficial effects.

Therefore, there is a need for the identification of new constituent compounds of maple syrup that could have beneficial effects on health.

There is a need for analogs of compounds from maple syrup that could have beneficial effects on health.

There is also a need for compositions containing new constituent compounds of maple syrup, and their analogs, that could have beneficial effects on health.

### SUMMARY

According to an embodiment, there is provided a compound of formula (I):

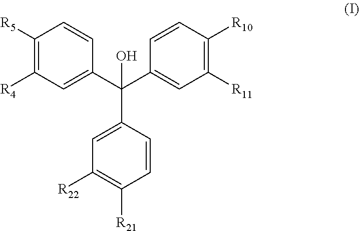

wherein

R₅, R₁₀, and R₂₁ may be OCH₃,

R₄, R₁₁, and R₂₂ may be independently chosen from OH, Cl, Br, and CHO,

pharmaceutically acceptable salt, racemic mixture, enantiomer, diastereoisomer, isomer, and tautomer thereof.

According to another embodiment, there is provided a compound of formula TRD6:

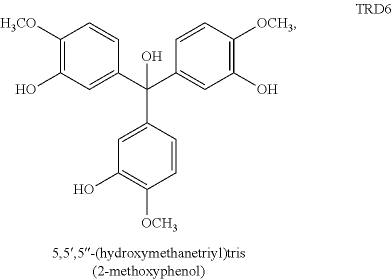 [SMILES: COc1ccc(C(O)(c2ccc(OC)c(O)c2)c2ccc(OC)c(O)c2)cc1O]

pharmaceutically acceptable salt, racemic mixture, enantiomer, diastereoisomer, isomer, and tautomer thereof.

According to another embodiment, there is provided a compound of formula TRD8:

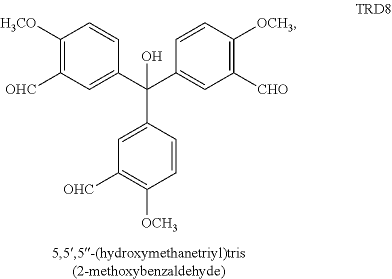 [SMILES: COc1ccc(C(O)(c2ccc(OC)c(C=O)c2)c2ccc(OC)c(C=O)c2)cc1C=O]

pharmaceutically acceptable salt, racemic mixture, enantiomer, diastereoisomer, isomer, and tautomer thereof.

According to another embodiment, there is provided a compound of formula TRD9:

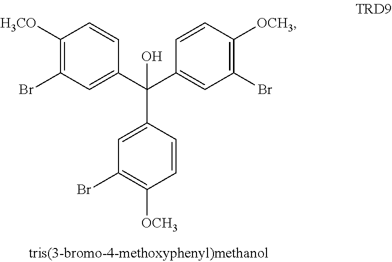 [SMILES: COc1ccc(C(O)(c2ccc(OC)c(Br)c2)c2ccc(OC)c(Br)c2)cc1Br]

pharmaceutically acceptable salt, racemic mixture, enantiomer, diastereoisomer, isomer, and tautomer thereof.

According to another embodiment, there is provided a compound of formula TRD10:

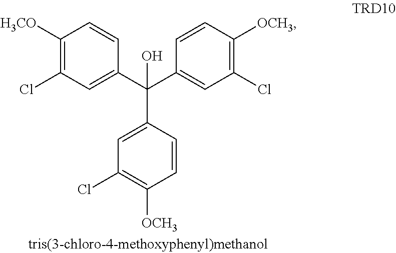 [SMILES: COc1ccc(C(O)(c2ccc(OC)c(Cl)c2)c2ccc(OC)c(Cl)c2)cc1Cl]

pharmaceutically acceptable salt, racemic mixture, enantiomer, diastereoisomer, isomer, and tautomer thereof.

According to another embodiment, there is provided a compound of formula QB12, QB48, QB49, QB56, and QB57:

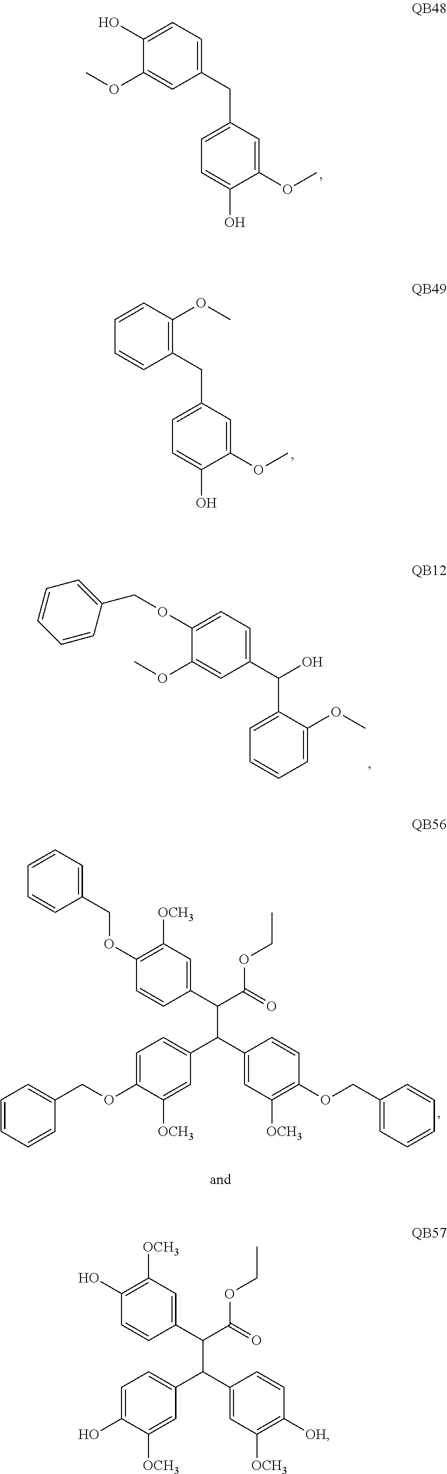

pharmaceutically acceptable salt, racemic mixture, enantiomer, diastereoisomer, isomer, and tautomer thereof.

According to another embodiment, there is provided a pharmaceutical composition comprising a therapeutically effective amount of a compound according to the present invention.

According to another embodiment, there is provided a method to inhibit tumor growth in a subject, which comprises administering a composition according to the present invention.

According to another embodiment, there is provided a method to inhibit tumor growth in a subject, which comprises administering an anticancer amount of a compound TRD1, RD5, TRD6, TRD7, TRD8, TRD9, TRD10, QB12, QB39, QB46, QB56, and QB57:

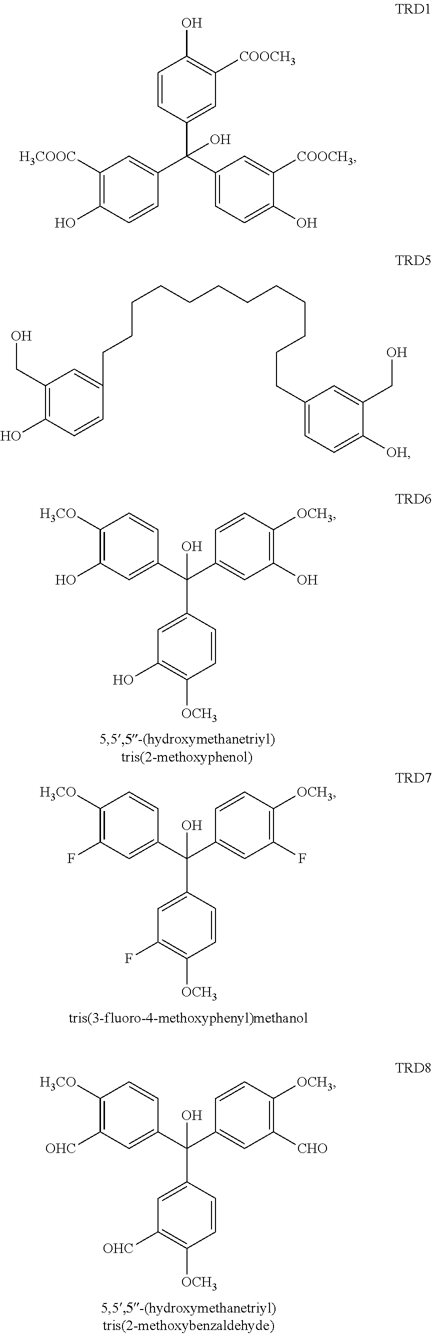

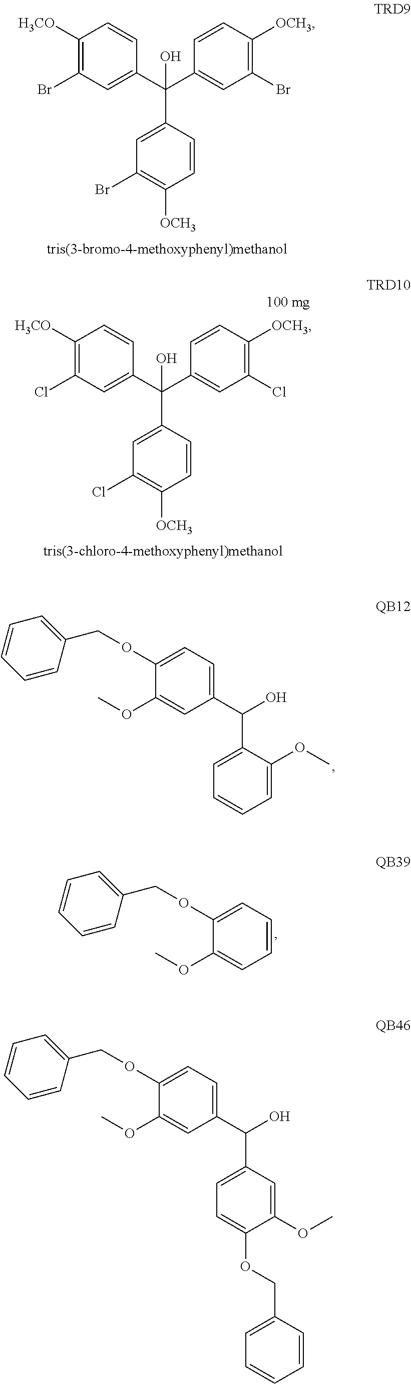 [SMILES: COc1ccc(C(O)(c2ccc(OC)c(Br)c2)c2ccc(OC)c(Br)c2)cc1Br ; COc1ccc(C(O)(c2ccc(OC)c(Cl)c2)c2ccc(OC)c(Cl)c2)cc1Cl ; COc1cc(C(O)c2ccccc2OC)ccc1OCc1ccccc1 ; COc1ccccc1OCc1ccccc1 ; COc1cc(C(O)c2ccc(OCc3ccccc3)c(OC)c2)ccc1OCc1ccccc1]

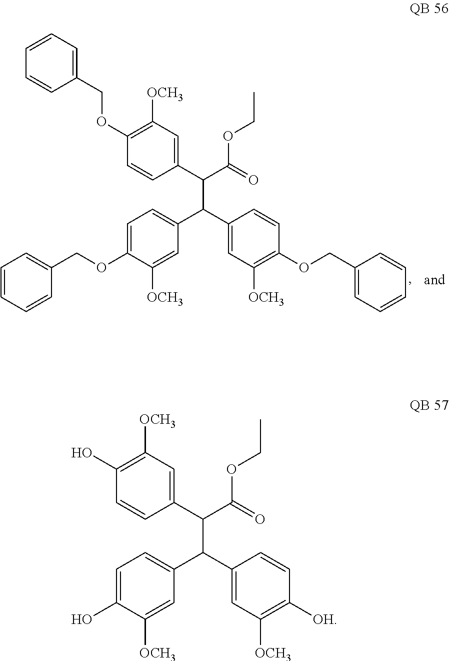 [SMILES: CCOC(=O)C(c1ccc(OCc2ccccc2)c(OC)c1)C(c1ccc(OCc2ccccc2)c(OC)c1)c1ccc(OCc2ccccc2)c(OC)c1 ; CCOC(=O)C(c1ccc(O)c(OC)c1)C(c1ccc(O)c(OC)c1)c1ccc(O)c(OC)c1]

According to another embodiment, there is provided a use of a compound of the present invention for the preparation of a medicament for the treatment of cancer.

According to another embodiment, there is provided a use of a compound of the present invention for the treatment of cancer.

According to another embodiment, there is provided a of a compound TRD1, TRD5, TRD6, TRD7, TRD8, TRD9, TRD10, QB12, QB39, QB46, QB56, and QB57 to inhibit tumor growth in a subject:

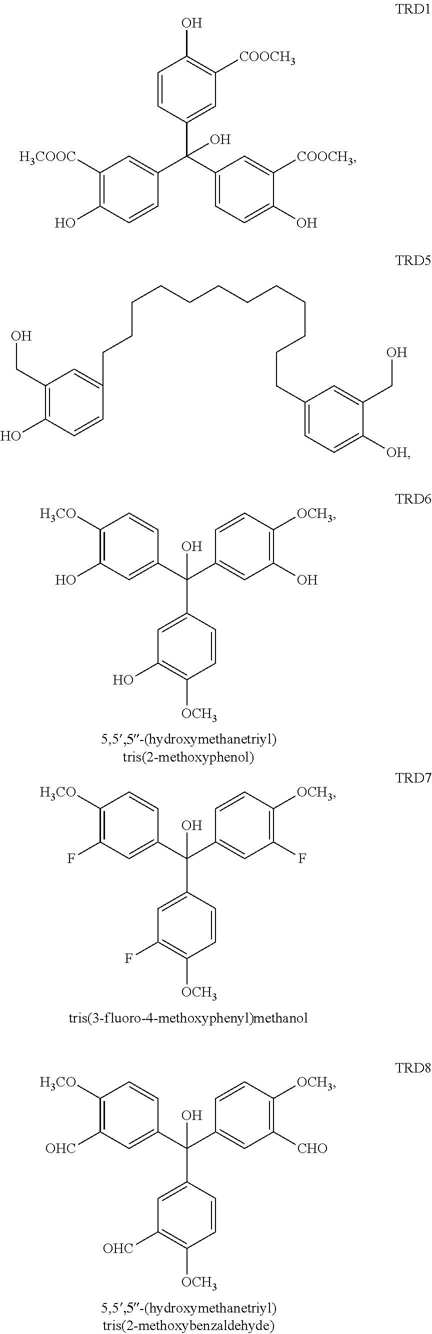

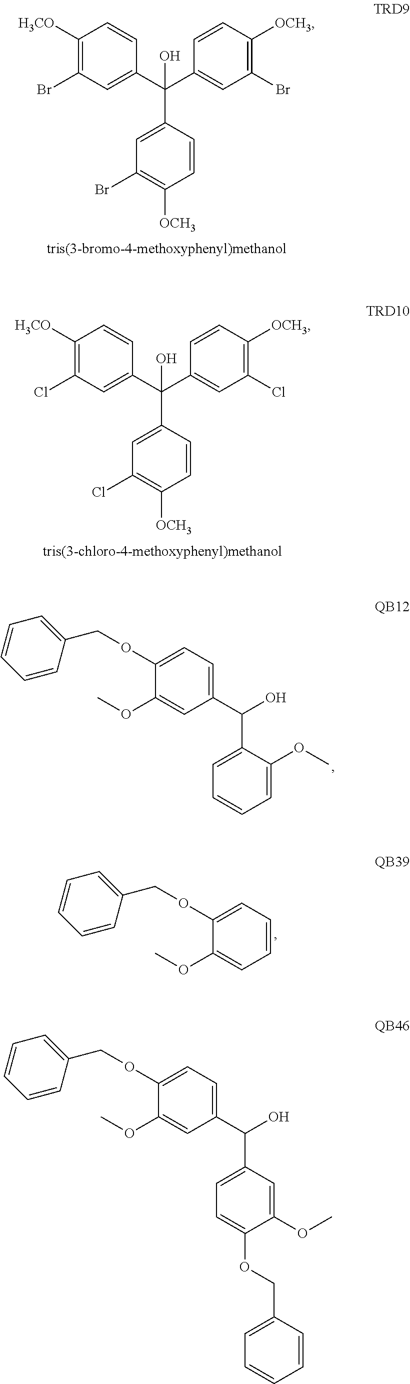 [SMILES: COc1ccc(C(O)(c2ccc(OC)c(Br)c2)c2ccc(OC)c(Br)c2)cc1Br ; COc1ccc(C(O)(c2ccc(OC)c(Cl)c2)c2ccc(OC)c(Cl)c2)cc1Cl ; COc1cc(C(O)c2ccccc2OC)ccc1OCc1ccccc1 ; COc1ccccc1OCc1ccccc1 ; COc1cc(C(O)c2ccc(OCc3ccccc3)c(OC)c2)ccc1OCc1ccccc1]

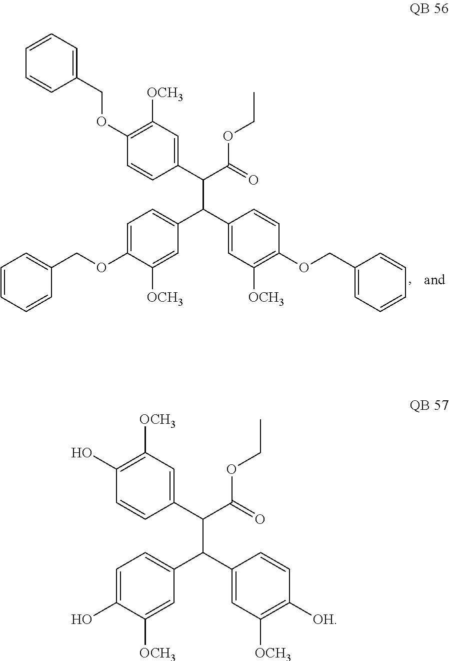 [SMILES: CCOC(=O)C(c1ccc(OCc2ccccc2)c(OC)c1)C(c1ccc(OCc2ccccc2)c(OC)c1)c1ccc(OCc2ccccc2)c(OC)c1 ; CCOC(=O)C(c1ccc(O)c(OC)c1)C(c1ccc(O)c(OC)c1)c1ccc(O)c(OC)c1]

According to another embodiment, there is disclosed a process for the synthesis of a compound of formula (5′):

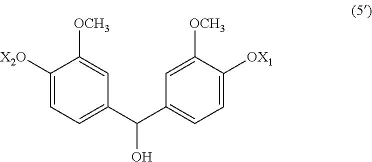

comprising the step of:

i. reacting a compound of formula (2′)

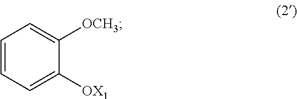

with a compound of formula (4′)

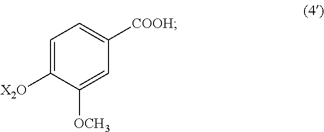

to obtain the compound of formula (5′)

wherein X₁, and X₂ may be a suitable protecting group for a hydroxyl group.

According to another embodiment, there is disclosed a process for the synthesis of a compound of formula (6′)

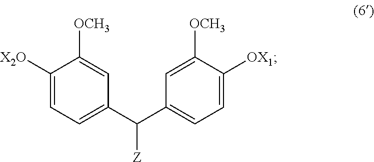

comprising the step of:

i. reacting a compound of formula (5′)

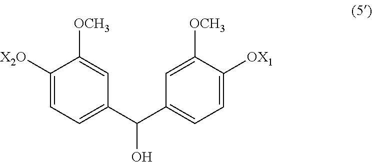

with a halogenating agent to obtain the compound of formula (6′),

wherein X₁, and X₂ may be a suitable protecting group for a hydroxyl group, and wherein Z may be a halogen atom.

According to another embodiment, there is disclosed a process for the synthesis of a compound of formula (8′)

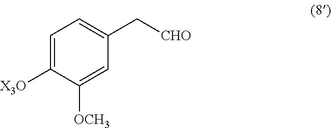

the process comprising the step of:

i. reacting a compound of formula (7′)

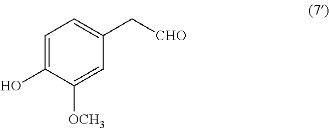 [SMILES: COc1cc(CC=O)ccc1O]

with a suitable hydroxyl protecting group, to obtain the compound of formula (8′)

wherein X₃ may be a suitable protecting group for a hydroxyl group; and

wherein Z may be a halogen atom.

According to another embodiment, there is disclosed a process for the synthesis of a compound of formula (9′)

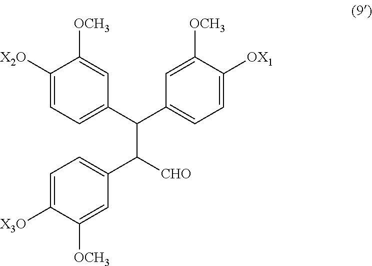

the process comprising the step of:

i. reacting a compound of formula (6′)

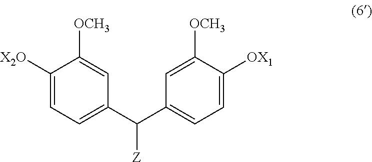

with a compound of formula (8′)

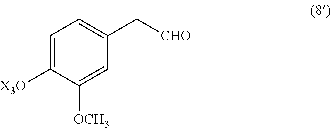

to obtain the compound of formula (9′),

wherein X₁, X₂ and X₃ may be a suitable protecting group for a hydroxyl group; and

wherein Z may be a halogen atom.

According to another embodiment, there is disclosed a process for the synthesis of a compound of formula (10′) (Quebecol)

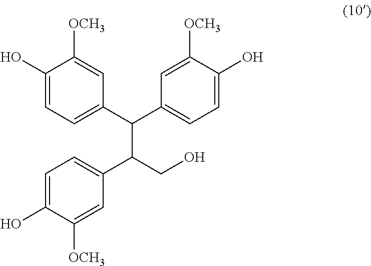 [SMILES: COc1cc(C(CO)C(c2ccc(O)c(OC)c2)c2ccc(O)c(OC)c2)ccc1O]

the process comprising the steps of:

i. reducing and deprotecting a compound of formula (9′)

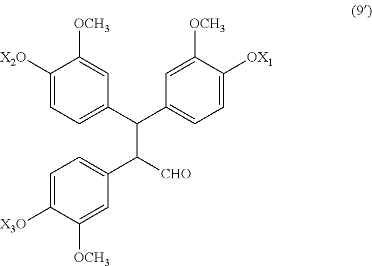

to obtain the compound of formula (10′) (Quebecol);

wherein X₁, X₂ and X₃ may be a suitable protecting group for a hydroxyl group; and

wherein Z may be a halogen atom

According to another embodiment, there is provided a process for the synthesis of a compound of formula (10′) (Quebecol)

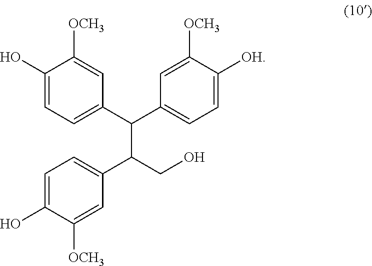 [SMILES: COc1cc(C(CO)C(c2ccc(O)c(OC)c2)c2ccc(O)c(OC)c2)ccc1O]

The process may be comprising the steps of:

i. reacting a compound of formula (1′)

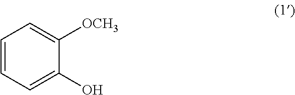

with a suitable hydroxyl protecting group, to obtain a compound of formula (2′)

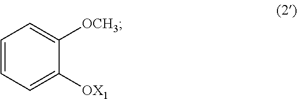

ii. reacting a compound of formula (3′)

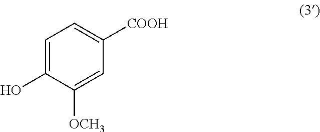 [SMILES: COc1cc(C(=O)O)ccc1O]

with a suitable hydroxyl protecting group, to obtain a compound of formula (4′)

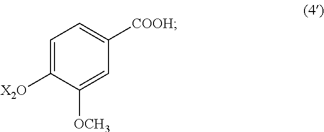

iii. reacting the compound of formula (2′) with the compound of formula (4′) to obtain a compound of formula (5′)

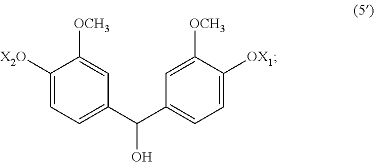

iv. reacting the compound of formula (5′) with a halogenating agent to obtain a compound of formula (6′)

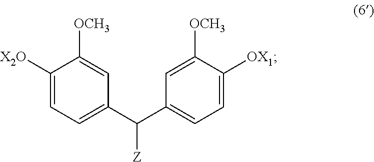

v. reacting a compound of formula (7′)

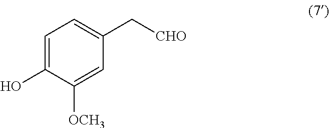 [SMILES: COc1cc(CC=O)ccc1O]

with a suitable hydroxyl protecting group, to obtain a compound of formula (8′)

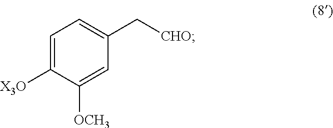

vi. reacting the compound of formula (6′) with the compound of formula (8′) to obtain a compound of formula (9′)

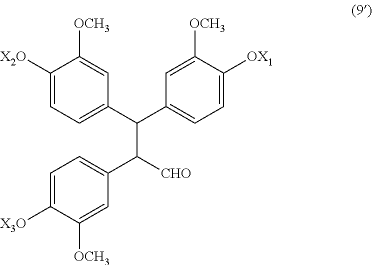

vii. reducing and deprotecting the compound of formula (9′) to obtain a compound of formula (10′) (Quebecol);

wherein X₁, X₂ and X₃ may be a suitable protecting group for a hydroxyl group; and wherein Z may be a halogen atom.

The X₃ may be chosen from Fluorenylmethyloxycarbonyl chloride (FMOC), Triphenylmethyl chloride, and a silyl ether.

The X₃ may be a silyl ether.

In the process according to the present invention, in step vii, the reducing may be by reacting the compound (9′) with NaBH₄.

In the process according to the present invention, the deprotection may be by reacting the compound of formula (9′) with one of tetra-n-butylammonium fluoride (TBAF) or trifluoroacetic acid (TFA).

In the process according to the present invention, the halogenating agent may be a trihalide of phosphorous and the trihalide of phosphorous may be chosen from PBr₃, and PCl₃.

According to another embodiment, there is provided a process for the synthesis of a compound of formula (3)

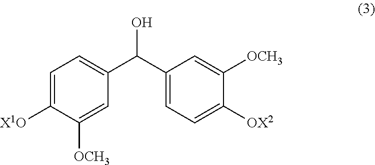

comprising the step of:

i. reacting a compound of formula (1)

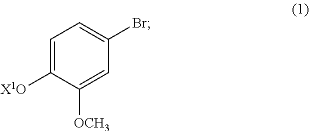

with a compound of formula (2)

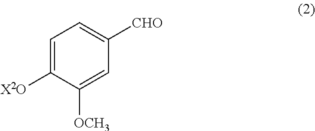

in presence of a strong base, to obtain a compound of formula (3)

wherein X¹ and X² may be a suitable protecting group for a hydroxyl group.

The strong base may be n-butyllithium (n-BuLi).

The reaction may be in tetrahydrofuran (THF) at −78° C.

According to another embodiment, there is provided a process for the synthesis of a compound of formula (4)

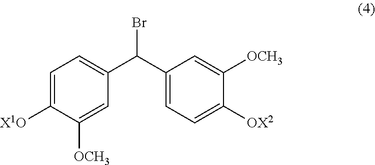

comprising the step of:

i. brominating a compound of formula (3)

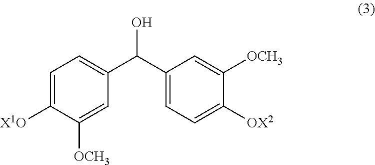

to obtain the compound of formula (4)

wherein X¹ and X² may be a suitable protecting group for a hydroxyl group.

The bromination may be with acetyl bromide (CH₃COBr).

The bromination may be with acetyl bromide (CH₃COBr) in benzene.

According to another embodiment, there is provided a process for the synthesis of a compound of formula (6)

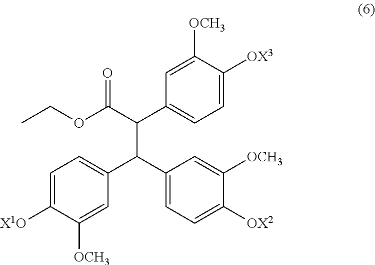

comprising the step of:

i. reacting a compound of formula (4)

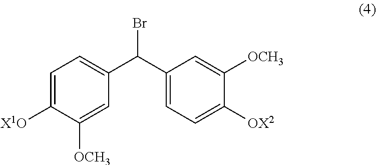

with a compound of formula (5)

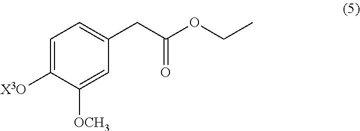

in the presence of a strong base, to obtain the compound of formula (6),

wherein X¹, X², and X³ may be a suitable protecting group for a hydroxyl group.

The strong base may be Lithium diisopropylamide (LDA).

The strong base may be Lithium diisopropylamide (LDA) in tetrahydrofuran at tetrahydrofuran (THF) at −78° C.

According to another embodiment, there is provided a process for the synthesis of a compound of formula (7)

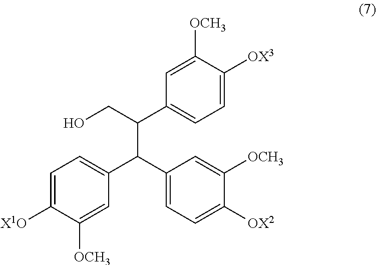

comprising the step of:

i. reducing a compound of formula (6)

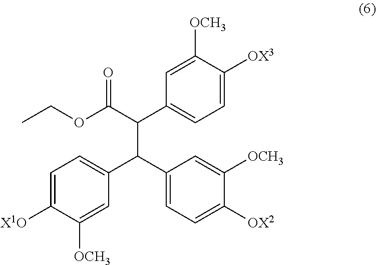

to obtain the compound of formula (7),

wherein X¹, X², and X³ may be a suitable protecting group for a hydroxyl group.

The reduction may be with lithium aluminum hydride (LiAlH₄).

The reduction may be with lithium aluminum hydride (LiAlH₄) in tetrahydrofuran (THF).

According to another embodiment, there is provided a process for the synthesis of a compound of formula (8) (Quebecol)

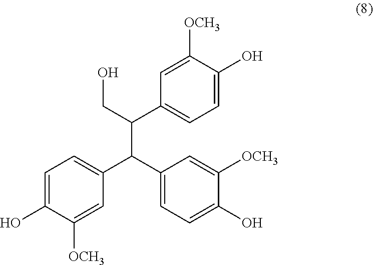 [SMILES: COc1cc(C(CO)C(c2ccc(O)c(OC)c2)c2ccc(O)c(OC)c2)ccc1O]

comprising the step of:

i. deprotecting a compound of formula (7)

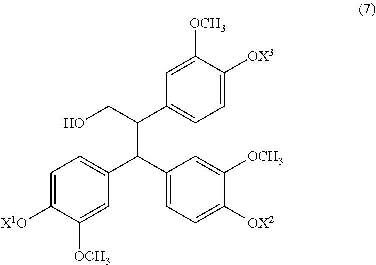

to obtain the compound of formula (8),

wherein X¹, X², and X³ may be a suitable protecting group for a hydroxyl group.

The deprotection may be with ammonium formate (HCO₂NH₄) and palladium on carbon (Pd/C).

The deprotection may be with ammonium formate (HCO₂NH₄) and palladium on carbon (Pd/C) in methanol (MeOH).

According to another embodiment, there is provided a process for the synthesis of a compound of formula (8) (Quebecol)

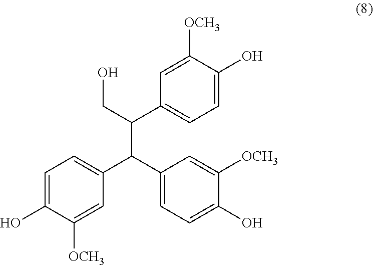 [SMILES: COc1cc(C(CO)C(c2ccc(O)c(OC)c2)c2ccc(O)c(OC)c2)ccc1O]

comprising the step of:
i. reacting a compound of formula (1)

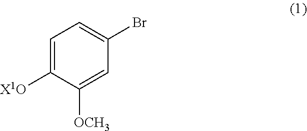

with a compound of formula (2)

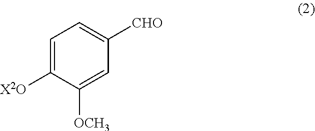

in presence of a strong base, to obtain a compound of formula (3)

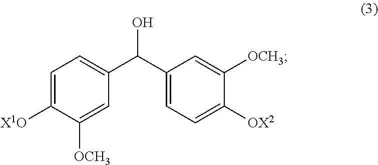

ii. brominating the compound of formula (3), to obtain a compound of formula (4)

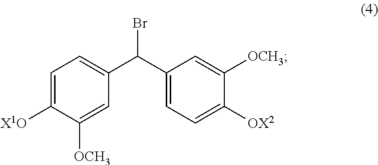

iii. reacting the compound of formula (4) with a compound of formula (5)

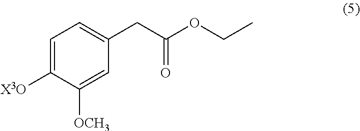

in the presence of a strong base, to obtain a compound of formula (6);
iv. reducing the compound of formula (6) to obtain a compound of formula (7)

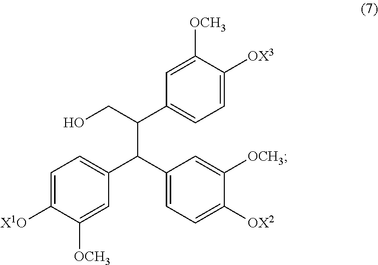

v. deprotecting the compound of formula (7) to obtain the compound of formula (8) (Quebecol),

wherein X¹, X², and X³ may be a suitable protecting group for a hydroxyl group.

The suitable protecting group for a hydroxyl group may be chosen from C₁-C₂₅ ethers, C₁-C₂₅ substituted methyl ethers, C₁-C₂₅ substituted ethyl ethers, C₁-C₂₅ acyl groups, C₁-C₂₅ halogenated acyl groups, C₁-C₂₅ substituted benzyl ethers, C₁-C₂₅ silyl ethers, C₁-C₂₅ esters, C₁-C₂₅ carbonates, and C₁-C₂₅ sulfonates.

The suitable protecting group for a hydroxyl group may be chosen from diphenylmethylchlorosilane (DPMS), Tosyl, methyl, methoxymethyl, benzyloxymethyl, tetrahydropyranyl, tetrahydrofuranyl, 2-(trimethylsilyl)ethoxymethyl, dioxanyl, 1-ethoxyethyl, 1-(2-chloroethoxy)ethyl, 2,2,2-trichloroethyl, t-butyl, allyl, propargyl, benzyl, p-methoxybenzyl, diphenylmethyl, triphenylmethyl, trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, triphenylsilyl, triisopropylsilyl, diphenylmethylsilyl, benzylformate, methylcarbonyl, ethylcarbonyl, methoxymethyl arbonyl, trichloroethoxycarbonyl, benzylcarbonyl, benzyloxycarbonyl. allylsulfonyl, methanesulfonyl, and p-toluenesulfonyl.

The suitable protecting group for a hydroxyl group may be benzyl (Bn).

In the process of the present invention, in step i), the strong base may be n-butyllithium (n-BuLi).

The reaction may be in tetrahydrofuran (THF) at −78° C.

In the process of the present invention, in step ii) brominating may be with acetyl bromide (CH₃COBr).

In the process of the present invention, in step ii) brominating may be with acetyl bromide (CH₃COBr) in benzene.

In the process of the present invention, in step iii) the strong base is Lithium diisopropylamide (LDA).

In the process of the present invention, in step iii) the strong base may be Lithium diisopropylamide (LDA) in tetrahydrofuran at tetrahydrofuran (THF) at −78° C.

In the process of the present invention, in step iv) reducing may be with lithium aluminum hydride (LiAlH₄).

In the process of the present invention, in step iv) reducing may be with lithium aluminum hydride (LiAlH₄) in tetrahydrofuran (THF).

In the process of the present invention, in step v) deprotecting may be with ammonium formate (HCO₂NH₄) and palladium on carbon (Pd/C).

The deprotection may be with ammonium formate (HCO₂NH₄) and palladium on carbon (Pd/C) in methanol (MeOH).

According to another embodiment, there is disclosed a compound of formula (5′) and (9′):

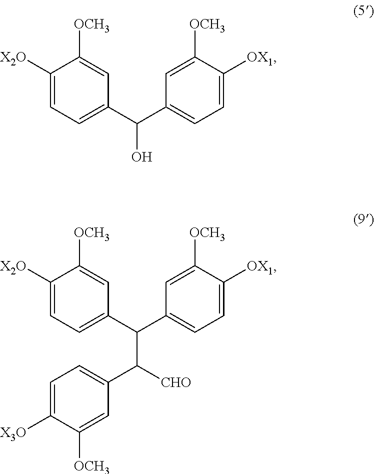

wherein X₁, X₂ and X₃ are as defined above.

According to another embodiment, there is disclosed a compound of formula (3), (4), (6) and (7):

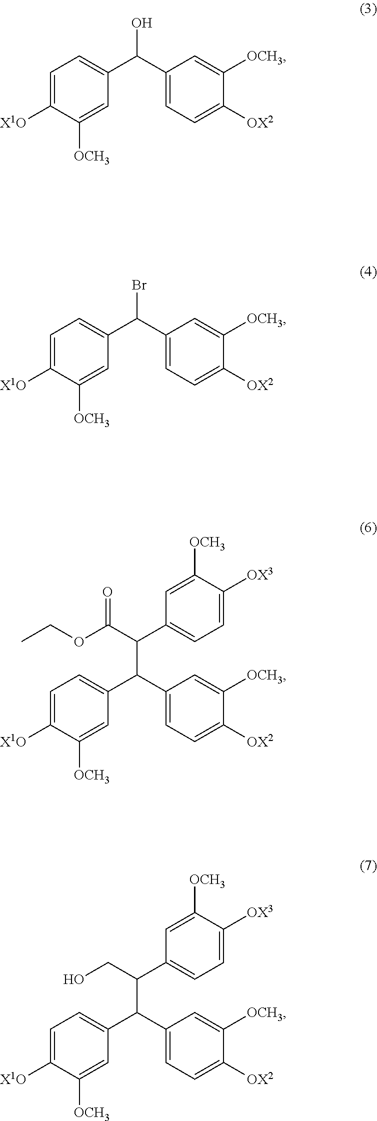

wherein X¹, X² and X³ are as defined above.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### BRIEF DESCRIPTION OF THE DRAWINGS

Further features and advantages of the present disclosure will become apparent from the following detailed description, taken in combination with the appended drawings, in which:

FIG. 1 illustrates the structural similarity between Tamoxifen and Quebecol.

FIG. 2 illustrates a reaction scheme for the synthesis of Quebecol according to an embodiment of the present invention.

FIG. 3 illustrates a reaction scheme for the synthesis of Quebecol according to an embodiment of the present invention

FIG. 4A illustrates a ¹H NMR spectrum of the compound 3.

FIG. 4B illustrates a MS spectrum of the compound 3.

FIG. 5A illustrates a ¹H NMR spectrum of the compound 6.

FIG. 5B illustrates a ¹H NMR spectrum of the compound 6.

FIG. 5C illustrates a ¹H NMR spectrum of the compound 6.

FIG. 5D illustrates a MS spectrum of the compound 6.

FIG. 6A illustrates a ¹H NMR spectrum of the compound 7.

FIG. 7B illustrates a ¹H NMR spectrum of the compound 7.

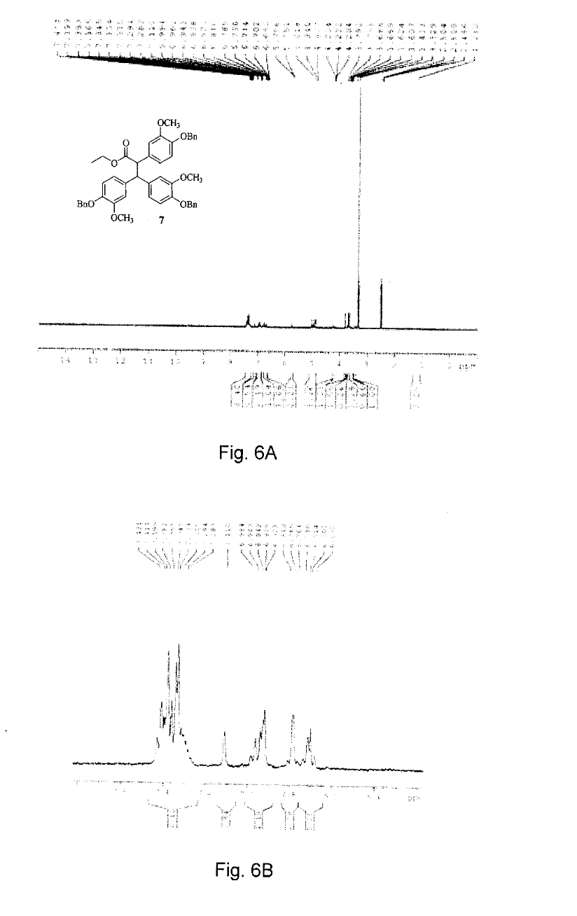

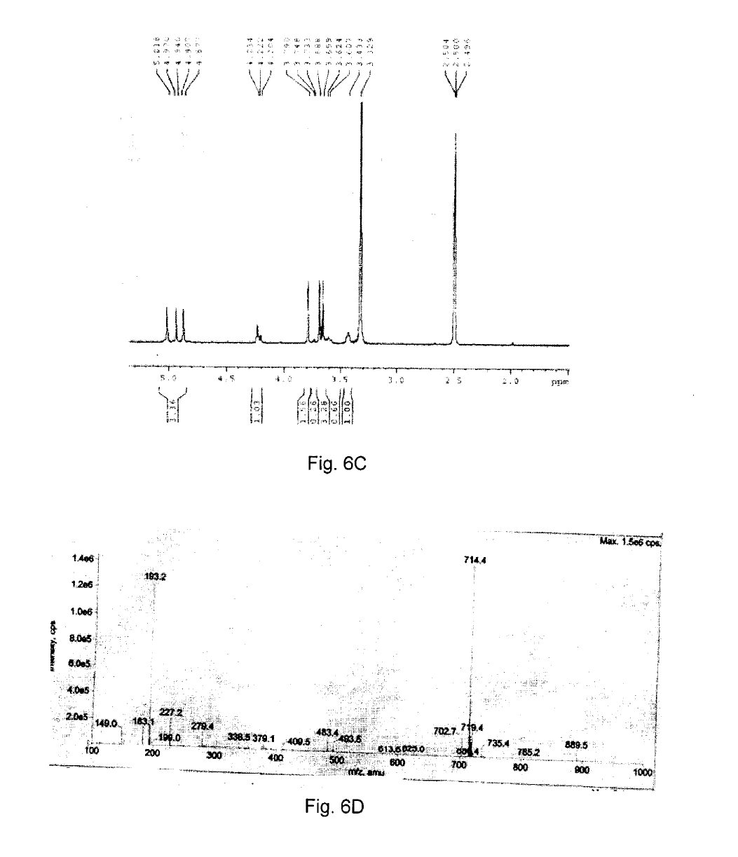

FIG. 6C illustrates a ¹H NMR spectrum of the compound 7.

FIG. 6D illustrates a MS spectrum of the compound 7.

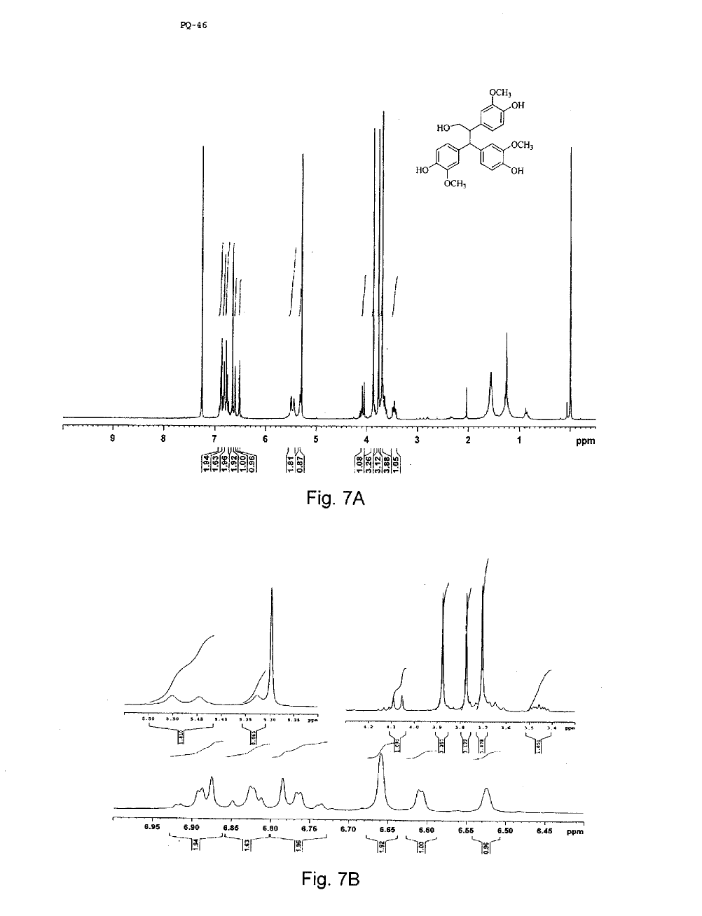

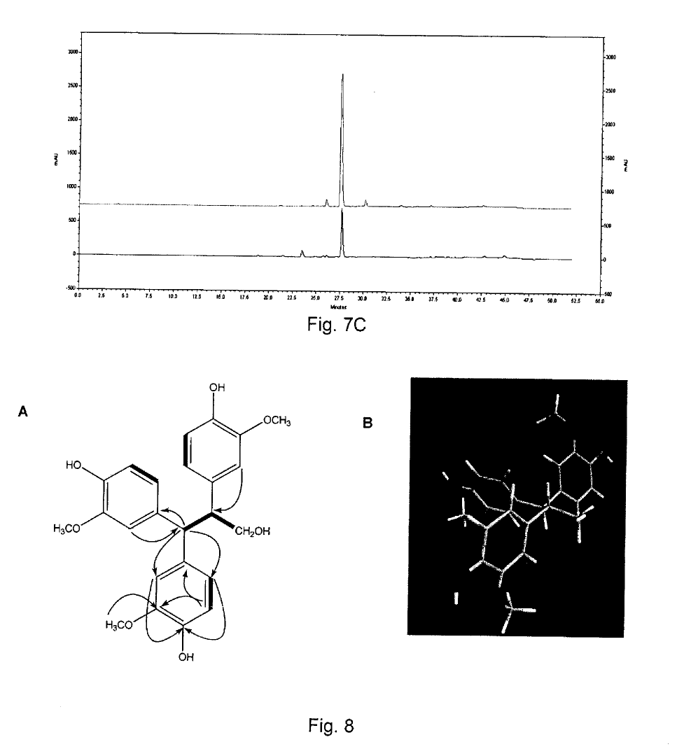

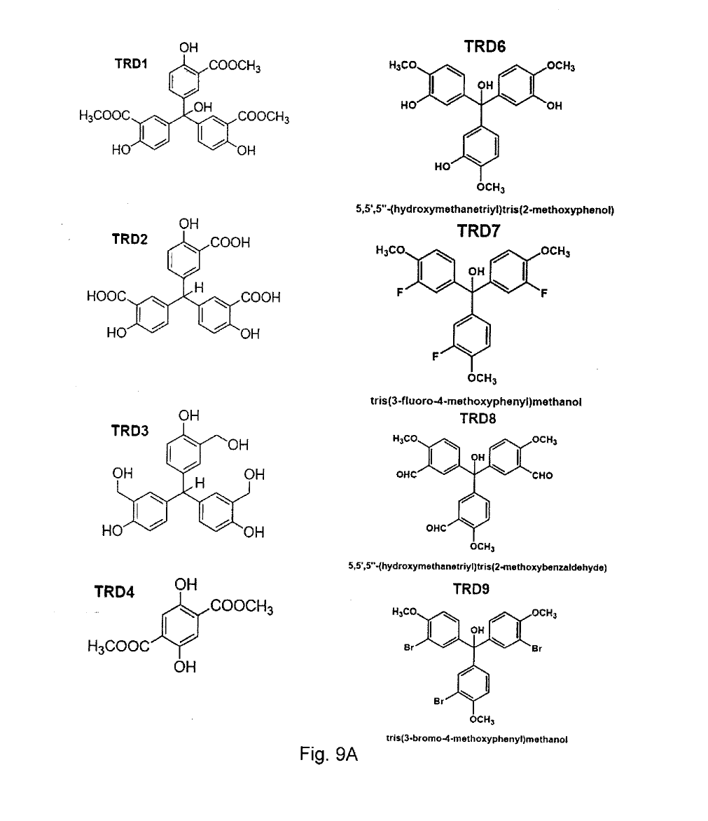

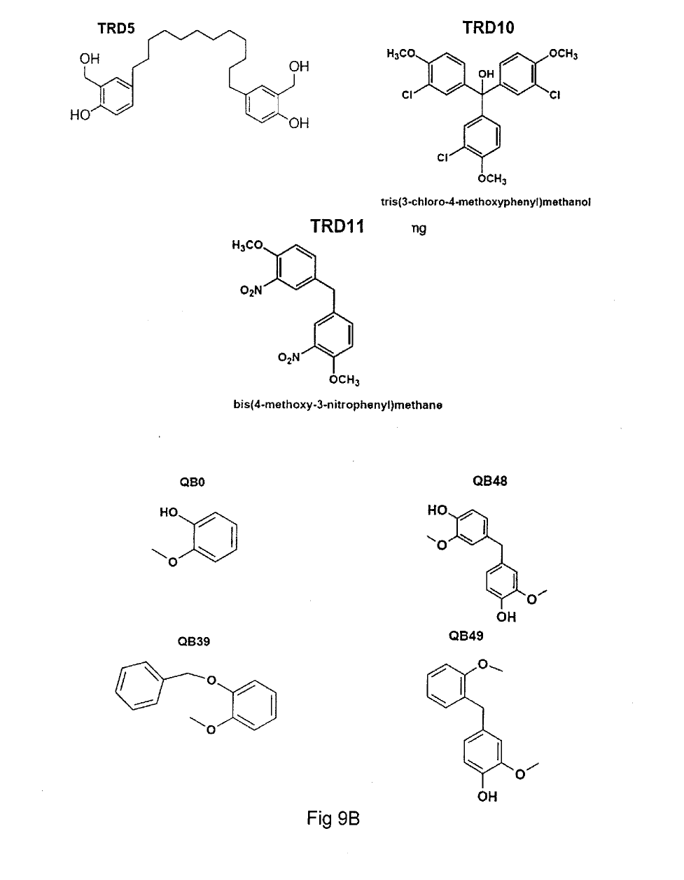

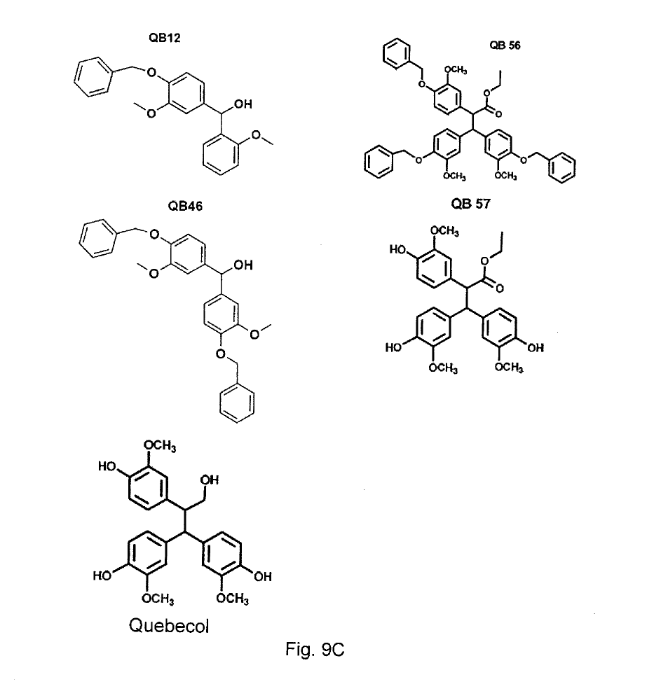

FIG. 7A illustrates a ¹H NMR spectrum of the compound 8-Quebecol.

FIG. 7B illustrates a ¹H NMR spectrum of the compound 8-Quebecol.

FIG. 7C illustrates a HPLC Chromatogram of natural Quebecol (bottom trace) vs. Synthetic Quebecol (top trace).

FIG. 8 illustrates the chemical structure of phenolic compound named Quebecol.

FIG. 9A illustrates the chemical structure of phenolic compounds that are derivatives of Quebecol.

FIG. 9B illustrates the chemical structure of phenolic compounds that are derivatives of Quebecol.

FIG. 9C illustrates the chemical structure of phenolic compounds that are derivatives of Quebecol.

### DETAILED DESCRIPTION OF THE PREFERRED EMBODIMENTS

In embodiments there is disclosed a new polyphenolic compound isolated from Canadian maple syrup. The compound, which is obtained as a pale yellow amorphous powder has been named Quebecol.

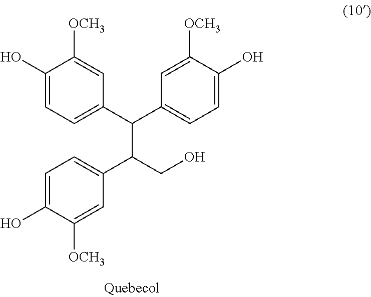 [SMILES: COc1cc(C(CO)C(c2ccc(O)c(OC)c2)c2ccc(O)c(OC)c2)ccc1O]

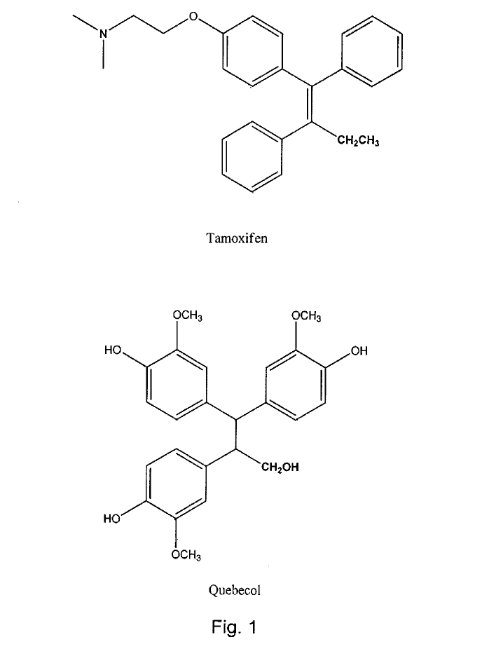

Now referring to FIG. 1, Quebecol displays some similarity to the known drug Tamoxifen. Tamoxifen is a widely used chemotherapy agent for hormonally dependent cancers such as breast cancer. However, Tamoxifen has severe side effects. Quebecol is a phytochemical derived compound present in maple syrup which has been consumed for centuries without toxicity. Thus, based on structural similarities to Tamoxifen and current laboratory assays, it is believed that Quebecol and analogs may exert greater anticancer effects than Tamoxifen without the adverse side effects.

According to another embodiment, the compounds of formula (I) are also represented by the compounds of formula (I):

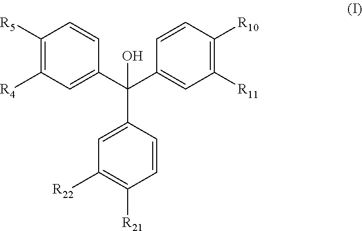

where R₅, R₁₀, and R₂₁ are OCH₃,

where R₄, R₁₁, and R₂₂ are independently chosen from OH, Cl, F, CF₃, CH₃Br, and CHO, and their pharmaceutically acceptable salts, racemic mixture, enantiomer, diastereoisomer, isomer, and tautomer thereof.

In embodiments, there is also disclosed compounds of formulae TRD6, TRD8, TRD9, and TRD10:

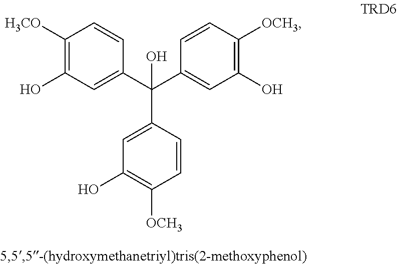 [SMILES: COc1ccc(C(O)(c2ccc(OC)c(O)c2)c2ccc(OC)c(O)c2)cc1O]

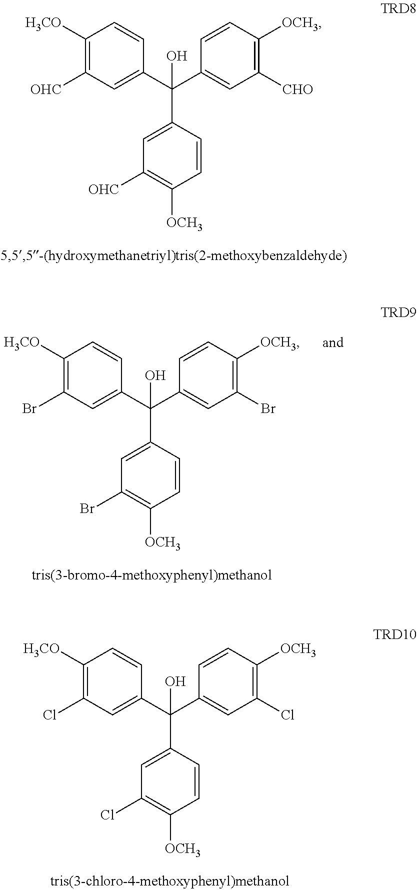 [SMILES: COc1ccc(C(O)(c2ccc(OC)c(C=O)c2)c2ccc(OC)c(C=O)c2)cc1C=O ; COc1ccc(C(O)(c2ccc(OC)c(Br)c2)c2ccc(OC)c(Br)c2)cc1Br ; "and" ; COc1ccc(C(O)(c2ccc(OC)c(Cl)c2)c2ccc(OC)c(Cl)c2)cc1Cl]

their pharmaceutically acceptable salt, racemic mixture, enantiomer, diastereoisomer, isomer, and tautomer thereof.

According to another embodiment, there is disclosed compounds of formulae QB12, QB48, QB49, QB56, and QB57:

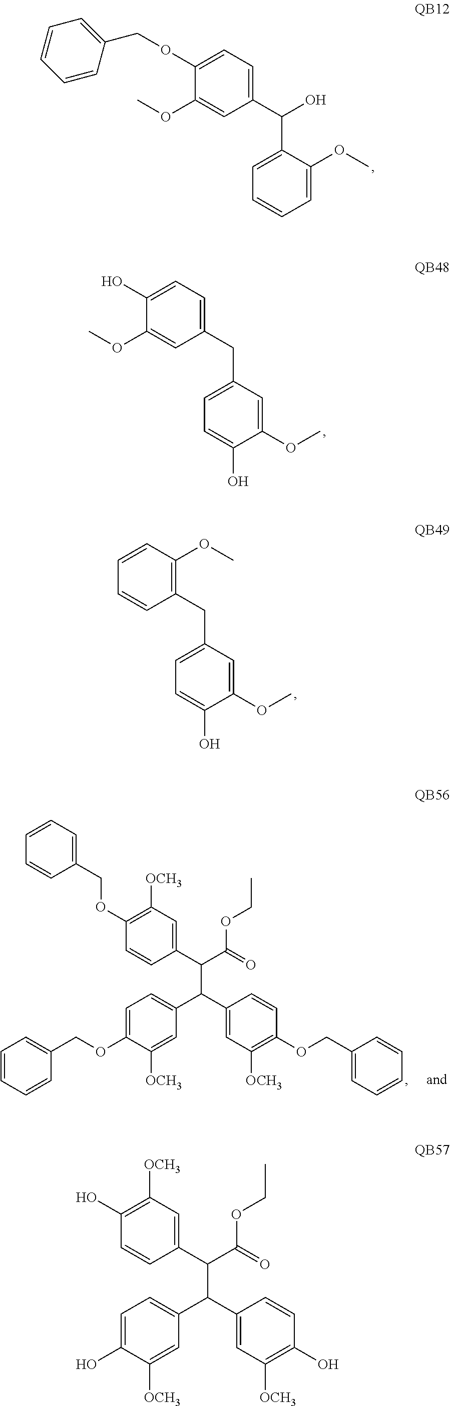

their pharmaceutically acceptable salt, racemic mixture, enantiomer, diastereoisomer, isomer, and tautomer thereof.

In embodiments, there is also disclosed a pharmaceutical composition comprising a therapeutically effective amount of a compound according to the present invention.

In embodiments, there is also disclosed a method to inhibit tumor growth in a subject, which comprises administering an anticancer amount of a compound of the present invention, or composition according to the present invention.

In embodiments, there is also disclosed method to inhibit tumor growth in a subject, which comprises administering an anticancer amount of a compound TRD1, TRD5, TRD6, TRD7, TRD8, TRD9, TRD10, QB12, QB39, QB46, QB56, and QB57:

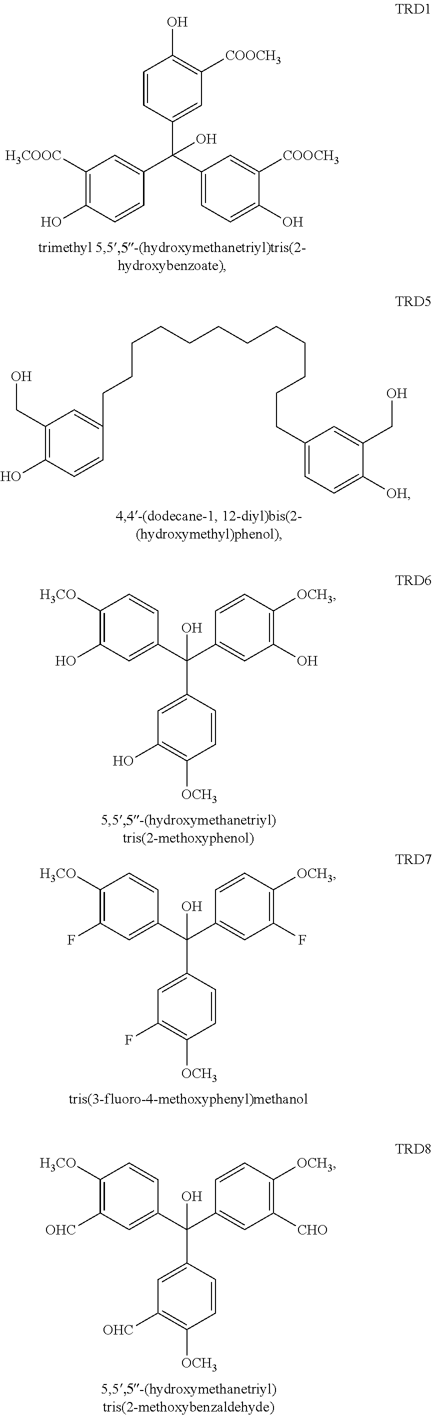

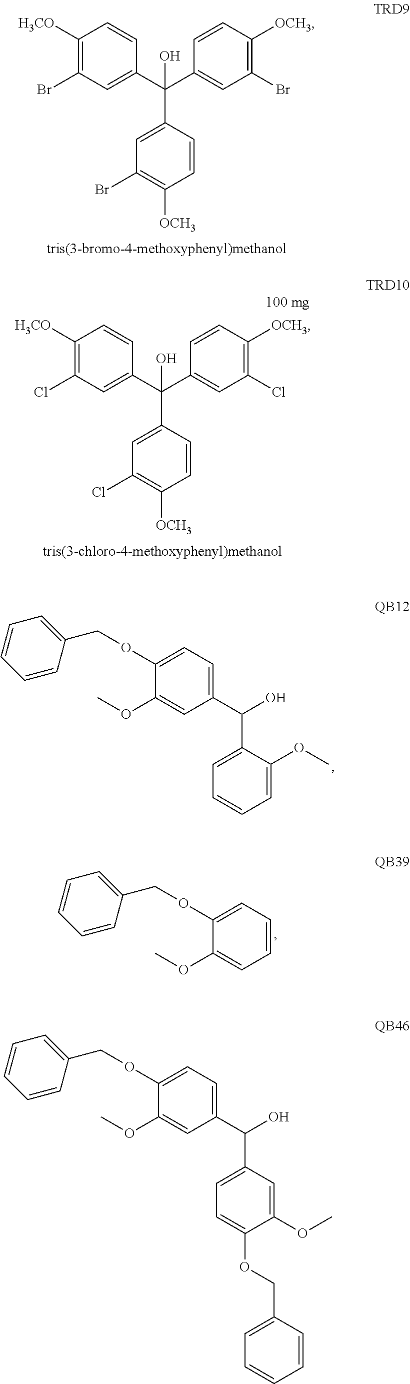 [SMILES: COc1ccc(C(O)(c2ccc(OC)c(Br)c2)c2ccc(OC)c(Br)c2)cc1Br ; COc1ccc(C(O)(c2ccc(OC)c(Cl)c2)c2ccc(OC)c(Cl)c2)cc1Cl ; COc1cc(C(O)c2ccccc2OC)ccc1OCc1ccccc1 ; COc1ccccc1OCc1ccccc1 ; COc1cc(C(O)c2ccc(OCc3ccccc3)c(OC)c2)ccc1OCc1ccccc1]

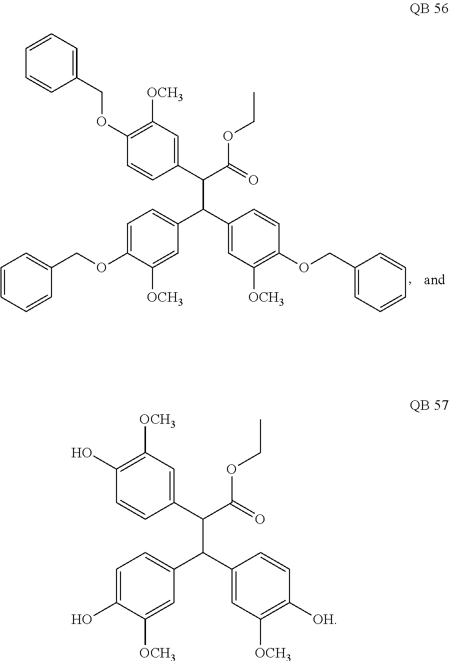 [SMILES: CCOC(=O)C(c1ccc(OCc2ccccc2)c(OC)c1)C(c1ccc(OCc2ccccc2)c(OC)c1)c1ccc(OCc2ccccc2)c(OC)c1 ; CCOC(=O)C(c1ccc(O)c(OC)c1)C(c1ccc(O)c(OC)c1)c1ccc(O)c(OC)c1]

In embodiments the tumor may be a breast tumor, a prostate tumor, a lung tumor, a colon tumor, a liver tumor and a testes tumor.

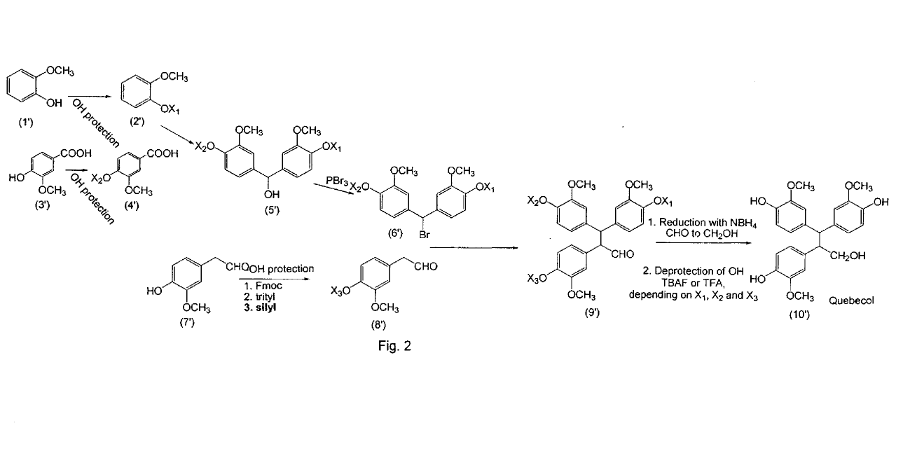

In embodiments, there is also disclosed a process for the synthesis of a compound of formula (10′) (Quebecol). Now referring to FIG. 2, the process comprises a first step of reacting a compound of formula (1′)

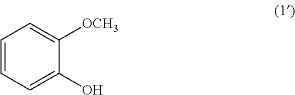

with a suitable hydroxyl protecting group, to obtain a compound of formula (2′)

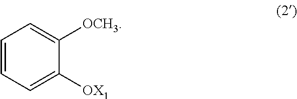

X₁ is a suitable protecting group for a hydroxyl group.

The process also comprises a second step of reacting a compound of formula (3′)

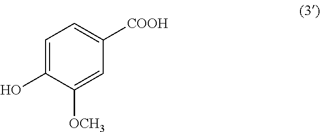 [SMILES: COc1cc(C(=O)O)ccc1O]

with a suitable hydroxyl protecting group, to obtain a compound of formula (4′)

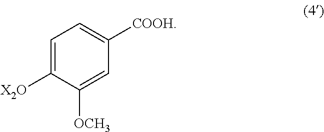

X₂ is a suitable protecting group for a hydroxyl group.

The third step of the process comprises the reaction of the compound of formula (2′) with the compound of formula (4′) to obtain a compound of formula (5′)

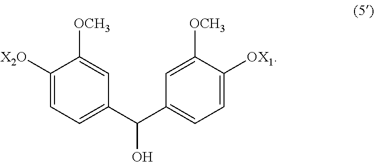

The fourth step of the process comprises reacting the compound of formula (5′) with a trihalide of phosphorus, such as phosphorus tribromide (PBr3), phosphorus trichloride (PCl₃), for example to obtain a compound of formula (6′)

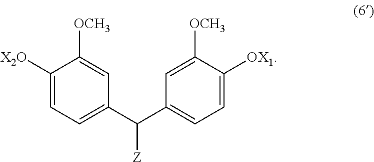

Z represents a halogen atom. Preferably, the halogen atom is Br.

The fifth step of the process comprises reacting the compound of formula (7′)

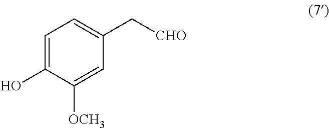 [SMILES: COc1cc(CC=O)ccc1O]

with a suitable hydroxyl protecting group, to obtain a compound of formula (8′)

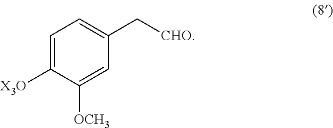

X₃ is a suitable protecting group for a hydroxyl group.

The sixth step of the process comprises the reaction of the compound of formula (6′) with the compound of formula (8′) to obtain a compound of formula (9′)

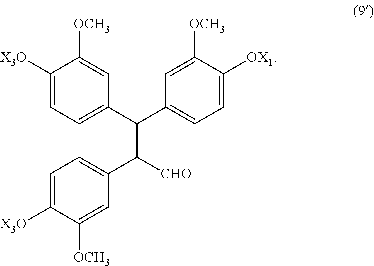

Finally, the seventh step of the process comprises reducing the CHO group to a CH₂OH group, and deprotecting the compound of formula (9′) to obtain a compound of formula (10′) (Quebecol). X₁, X₂ and X₃ represent suitable protecting groups for a hydroxyl groups.

The suitable protecting groups for hydroxyl groups for X₃ may be chosen from FMOC, triphenylmethyl chloride, and a silyl ether. Preferably, the protecting group is a silyl ether protecting group.

According to an embodiment of the present invention, the reduction reaction of the compound of formula (9′) may be effected with NaBH₄.

The deprotection of the compound of formula (9′) may be achieved with one of tetra-n-butylammonium fluoride (TBAF) or trifluoroacetic acid (TFA), depending on the protecting group for a hydroxyl group chosen.

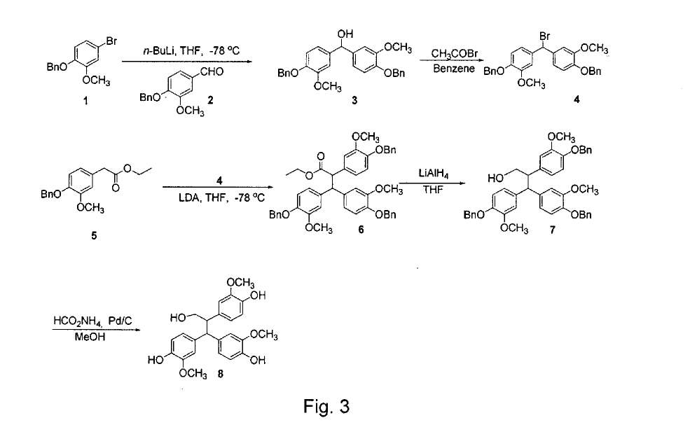

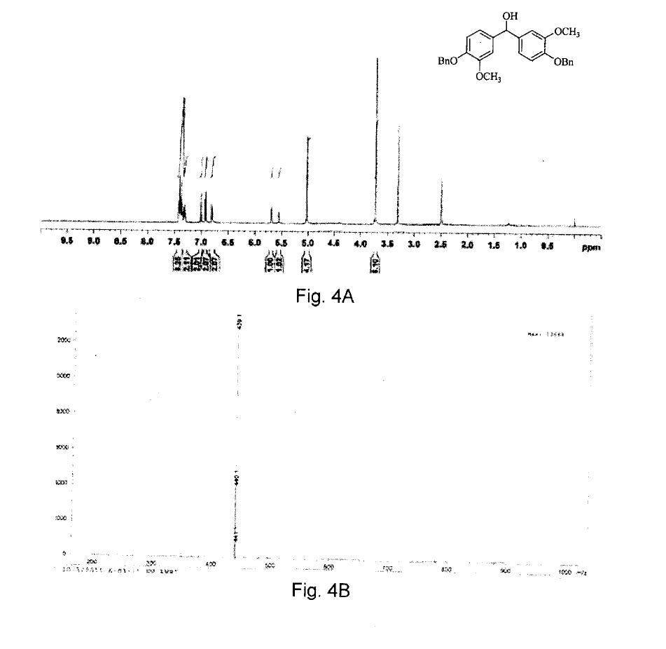

In embodiments, there is also disclosed an alternative process for the synthesis of a compound of formula (8) (Quebecol). Now referring to FIG. 3, the process comprises a first step of reacting a compound of formula (1)

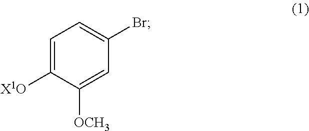

with a compound of formula (2)

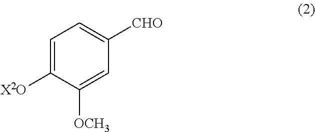

in presence of a strong base, to obtain a compound of formula (3)

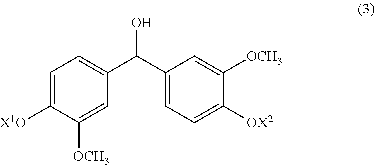

wherein X¹ and X² is a suitable protecting group for a hydroxyl group. According to an embodiment, the strong base may be for example n-butyllithium (n-BuLi). The reaction may take place for example in tetrahydrofuran (THF) at −78° C.

The second step of the process involves brominating a compound of formula (3)

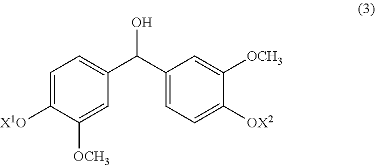

to obtain the compound of formula (4)

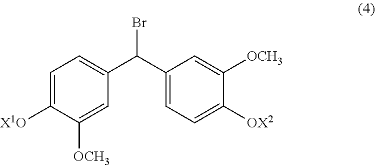

where X¹ and X² is a suitable protecting group for a hydroxyl group. Bromination is preferably done with acetyl bromide (CH₃COBr). The reaction may be carried out for example in benzene.

The third step involves reacting a compound of formula (4)

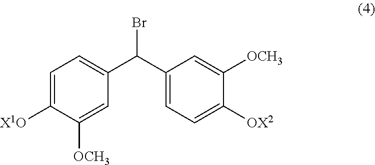

with a compound of formula (5)

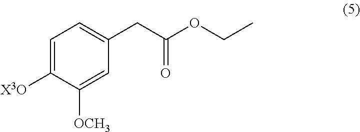

in the presence of a strong base, to obtain a compound of formula (6),

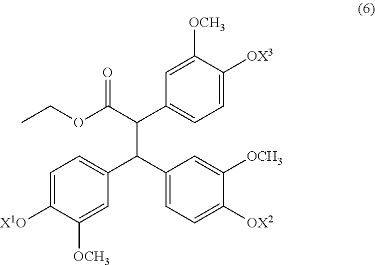

where X¹, X², and X³ is a suitable protecting group for a hydroxyl group. The strong base may be for example lithium diisopropylamide (LDA). The reaction may be carried out in tetrahydrofuran (THF) at −78° C. for example.

The fourth step involves reducing a compound of formula (6)

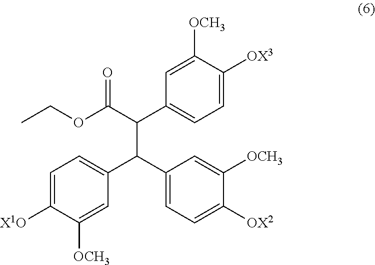

to obtain the compound of formula (7),

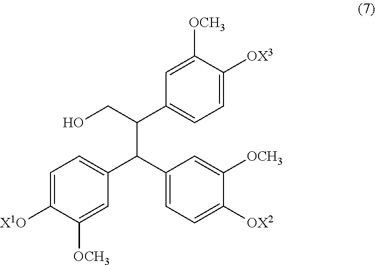

where X¹, X², and X³ is a suitable protecting group for a hydroxyl group. The reduction may be achieved for example with lithium aluminum hydride (LiAlH₄). The reaction may be carried out in tetrahydrofuran (THF).

The fifth step involves deprotecting a compound of formula (7)

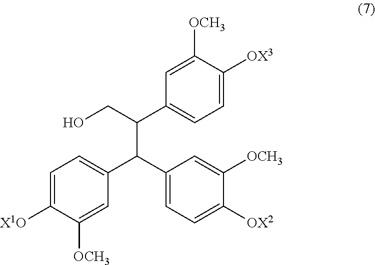

to obtain the compound of formula (8),

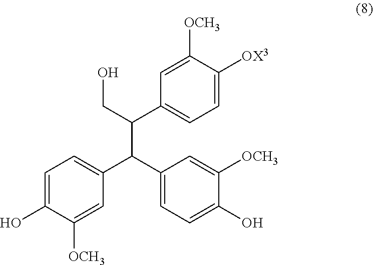

where X¹, X², and X³ is a suitable protecting group for a hydroxyl group. The deprotection may be achieved for example with ammonium formate (HCO₂NH₄) and palladium on carbon (Pd/C). The reaction may be carried out in methanol, for example.

Suitable protecting group for a hydroxyl group include but are not limited to C₁-C₂₅ ethers, C₁-C₂₅ substituted methyl ethers, C₁-C₂₅ substituted ethyl ethers, C₁-C₂₅ acyl groups, C₁-C₂₅ halogenated acyl groups, C₁-C₂₅ substituted benzyl ethers, C₁-C₂₅ silyl ethers, C₁-C₂₅ esters, C₁-C₂₅ carbonates, and C₁-C₂₅ sulfonates. Other suitable protecting group for a hydroxyl group include but are not limited to diphenylmethylchlorosilane (DPMS), tosyl, methyl, methoxymethyl, benzyloxymethyl, tetrahydropyranyl, tetrahydrofuranyl, 2-(trimethylsilyl)ethoxymethyl, dioxanyl, 1-ethoxyethyl, 1-(2-chloroethoxy)ethyl, 2,2,2-trichloroethyl, t-butyl, allyl, propargyl, benzyl, p-methoxybenzyl, diphenylmethyl, triphenylmethyl, trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, triphenylsilyl, triisopropylsilyl, diphenylmethylsilyl, benzylformate, methylcarbonyl, ethylcarbonyl, methoxymethyl carbonyl, trichloroethoxycarbonyl, benzylcarbonyl, benzyloxycarbonyl, allylsulfonyl, methanesulfonyl, and p-toluenesulfonyl.

Preferably, the suitable protecting group for a hydroxyl group is benzyl (Bn).

The present invention will be more readily understood by referring to the following examples which are given to illustrate the invention rather than to limit its scope.

### Example 1

### Identification of a new compound from the process of preparation of Maple Syrup

Reagents & Materials:

All solvents are either analytical grade or HPLC grade and purchased from Wilkem Scientific Co. (Pawtucket, R.I.). Maple syrup (grade C, 20 L) is provided by the Federation of Maple Syrup Producers of Quebec (Canada). The syrup is kept frozen until extraction when it is subjected to liquid-liquid partitioning with ethyl acetate (10 L×3) followed by n-butanol (10 L×3) solvents, to yield ethyl acetate (4.7 g) and butanol (108 g) extracts, respectively, after solvent removal in vacuo.

Isolation:

A portion of the butanol extract (87 g) is reconstituted in methanol to afford methanol soluble (36 g) and insoluble (57 g) fractions. The methanol soluble fraction is selected for further purification by repeated Sephadex-LH2O column chromatography followed by C 18 semi-preparative HPLC. First, the extract is chromatographed on 65×4 cm Sephadex-LH-20 column eluted with a CH₃OH—H₂O gradient system (3:7 to 1:0, v/v) to afford twelve subfractions, A1-A12. Subfraction A4 (1.6 g) is re-chromatographed on a 65×4 cm Sephadex-LH-20 column eluted with same gradient system (3:7 to 1:0, v/v) to afford twelve subfractions, B1-B12. Subfraction B5 (137.2 mg) is purified by semi-preparative HPLC (Neckman Coulter) using a Waters Sunfire C18 column (250×10 mm i.d., 5 μm, flow=2 mL/min) with a gradient elution system of CH₃OH—H₂O (0.1% trifluoroacetic acid) (1:4, v/v to 1:0, v/v in 60 min) to afford compound 1 (4 mg).

NMR:

Data is collected on a Varian 500 MHz Biospin instrument using CD₃OD as solvent.

Compound (10)—Quebecol, (FIGS. 1 and 8) is obtained as pale yellow amorphous powder. The positive ESIMS exhibits a molecular peak at m/z 449.1571 [M+Na]⁺, and negative ESI shows at m/z 425.1979 [M-H]⁻. The ¹H NMR (in DMSO-d₆) spectrum exhibits the signals for three pairs of ABX aromatic system at δ_{H} 6.81 (1H, J=8.0 Hz, H-6), 6.67 (1H, J=8.0 Hz, H-5), 6.98 (1H, s, H-2); 6.56 (1H, J=8.0 Hz, H-6′), 6.41 (1H, J=8.0 Hz, H-5′), 6.78 (1H, s, H-2′); 6.60 (1H, J=8.0 Hz, H-6″), 6.50 (1H, J=8.0 Hz, H-5″), 6.56 (1H, s, H-2″) respectively, suggesting the presence of three benzene rings, which is supported by the ¹³C NMR (in DMSO-d₆) data (Table 4) and ¹H—¹H COSY spectrum analysis (FIG. 4). Three singlet signals at δ_{H} 3.76, 3.66 and 3.63 with three-proton density for each reveal the presence of three methoxyl groups. Additionally, one doublet signal at δ_{H} 4.02 (1H, J=10.5 Hz, H-7), two multiplet signals at d_{H} 3.41 (1H, m, H-8) and 3.40 (2H, m, H-10) can be observed in the ¹H spectrum. All the proton signals are assigned to the corresponding carbons through direct ¹H—¹³C correlations in the HSQC (Table 4) spectrum, with exception of the two singlets at δ_{H} 8.67 (1H) and 8.43 (2H) which are in good accordance with proton of hydroxyl group. Furthermore a CH—CH—CH2 substructure can be deduced from COSY correlations (FIG. 4) analysis. In the HMBC spectrum, the correlations signals (FIG. 4) from δ_{H} 6.67 (H-5) and 3.76 (3-OCH₃) to C-3 (δ 147.72), δ_{H} 6.41 (H-5′) and 3.66 (3′-OCH₃) to C-3′ (δ 147.17), δ_{H} 6.50 (H-5″) and 3.63 (3″-OCH₃) to C-3″ (δ 147.08), reveals three methoxyl groups substituted on the C-3, 3′ and 3″ individually. In the same HMBC experiment, correlation signals show from δ_{H} 4.02 (H-7) to C-2 (112.56), C-6 (120.33) and C-1′ (136.26), and from δ_{H} 6.78 (H-2′) to C-8 (51.42) suggest three benzene rings are attached to the CH—CH—CH₂OH chain on C-7, C-7 and C-8 position respectively.

TABLE 1
¹H and ¹³C NMR data (in DMSO-d6,
500 and 125 MHz) of compound 10

| No | δ_{C} | δ_{H} (J in Hz) | No | δ_{C} | δ_{H} (J in Hz) |
|---|---|---|---|---|---|
| 1 | 136.70 | — | 1′ | 136.26 | — |
| 2 | 112.56 | 6.98 (s) | 2′ | 113.15 | 6.78 (s) |
| 3 | 147.72 | — | 3′ | 147.17 | — |
| 4 | 144.92 | — | 4′ | 144.26 | — |
| 5 | 115.72 | 6.67 (d, 8.0) | 5′ | 115.23 | 6.41 (d, 8.0) |
| 6 | 120.33 | 6.81 (d, 8.0) | 6′ | 121.04 | 6.56 (d, 8.0) |
| 7 | 52.67 | 4.02 (d, 10.5) | 1″ | 134.65 | — |
| 8 | 51.42 | 3.41 (m) | 2″ | 113.90 | 6.78 (s) |
| 9 | 64.92 | 3.40 (m) | 3″ | 147.08 | — |
| 3-OCH3 | 56.14 | 3.76 (s) | 4″ | 144.48 | — |
| 3′-OCH3 | 56.01 | 3.66 (s) | 5″ | 115.09 | 6.50 (d, 8.0) |
| 3″-OCH3 | 55.94 | 3.63 (s) | 6″ | 121.77 | 6.60 (d, 8.0) |
| 4-OH | — | 8.64 (s) | 4″-OH | — | 8.43 (s) |
| 4′-OH | — | 8.43 (s) | | | |

The absolute configuration of compound (10) is elucidated by combination of ¹H NMR analysis and computer modelling. The coupling constant of H-7 is 10.5 Hz, suggesting H-7 and H-8 are both at the axial positions, which is in accordance with S configuration. Thus, based on above findings, the structure of compound (10) is elucidated as shown in FIG. 4 to which the common name, Quebecol, has been assigned.

### Example 2

### Preparation of a Compound of Formula (2′)

### Reaction Scheme of Example 2

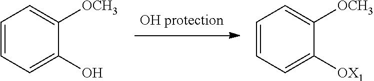

Compounds of formula (2′) may be synthesized, for example, by using the following conditions. To a stirred solution of the corresponding commercially available phenolic compound (1.00 mmol) in acetone is added potassium carbonate (1.50 mmol) and benzyl bromide (1.10 mmol). The solution was then stirred at ambient temperature (˜30° C.) for 16 h. The organic solvent was evaporated under reduced pressure. The residue was diluted with water and extracted with ethyl acetate. The organic layer was dried over anhydrous Na₂SO₄ and evaporated under reduced pressure. The crude compound was purified by column chromatography (ethyl acetate/hexane) to afford corresponding benzylated compound in 80-95% yields.

### Example 3

### Preparation of a Compound of Formula (4′)

### Reaction Scheme of Example 3

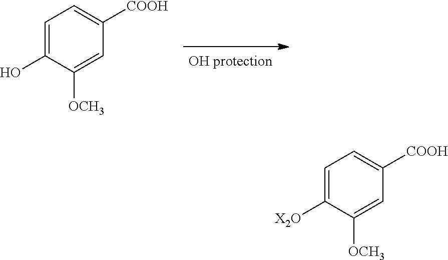

Compounds of formula (4′) may be synthesized, for example, by using the following conditions. To a stirred solution of the corresponding commercially available phenolic compound (1.00 mmol) in acetone is added potassium carbonate (1.50 mmol) and benzyl bromide (1.10 mmol). The solution was then stirred at ambient temperature (˜30° C.) for 16 h. The organic solvent was evaporated under reduced pressure. The residue was diluted with water and extracted with ethyl acetate. The organic layer was dried over anhydrous Na₂SO₄ and evaporated under reduced pressure. The crude compound was purified by column chromatography (ethyl acetate/hexane) to afford corresponding benzylated compound in 80-95% yields.

### Example 4

### Preparation of a Compound of Formula (5′)

### Reaction Scheme of Example 4

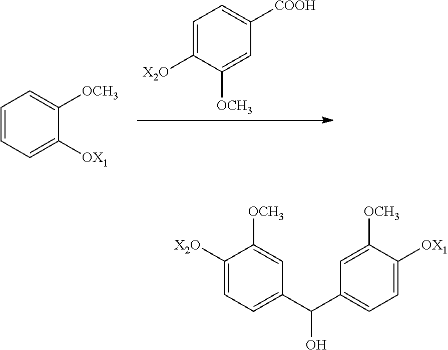

Compound (2′) (80 mmol) was reacted with 4′ (80 mmol) in the presence of polyphosphoric acid at 80° C. for 30 min according to the previously reported procedure (Harig et al., Eur. J. Org. Chem. 2004, 2381-2397). The crude product (80 mmol) was suspended in methanol (400 mL), which had been deacidified by passing it through basic alumina. Pyridine (0.5 mL) and palladium on charcoal (10% Pd, oxidic form) were then added and the mixture was shaken under hydrogen in a hyderogenerator. The suspension was then filtered through silica gel and the filter was washed with deacidified methanol. Removal of the solvent under reduced pressure afforded 5′.

### Example 5

### Preparation of a Compound of Formula (6′)-bis(4-(Benzyloxy)-3-methoxyphenyl)bromomethane

### Reaction Scheme of Example 5

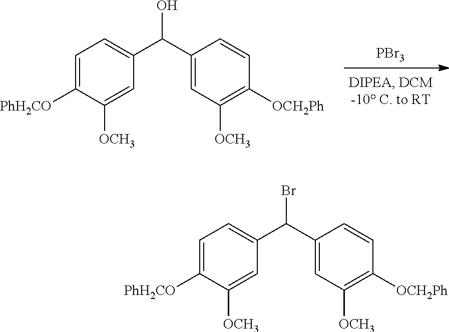

Bis(4-(benzyloxy)-3-methoxyphenyl)methanol (0.68 g, 1.5 mmol) was dissolved in dry DCM (20 mL) and then to the solution was added N,N-diisopropylethylamine (347 μl, 2.0 mmol). The mixture was cooled to −10° C. PBr₃ (176 μl, 1.1 eq.) in dry DCM (10 mL) was added dropwise in the dark over 15 min. The reaction mixture was brought to 0° C. and was stirred for 1 h and then it was stirred at room temperature for additional 6 h. After completion of the reaction as indicated by TLC, the reaction mixture was concentrated on rotatory evaporator under reduced pressure. The residue was washed with water (10 mL) and extracted with ethyl acetate (2×10 mL). The combined organic phases were dried over anhydrous sodium sulfate and concentrated. The product was purified by column chromatography over silica gel as a white solid showing a mixture of brominated compound and ketone product.

### Example 6

### Preparation of a Compound of Formula (8′)

### Reaction Scheme of Example 6

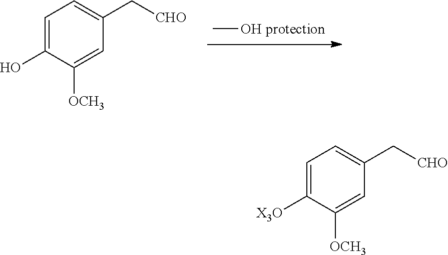

Compounds of formula (8′) may be synthesized, for example, by using the following conditions. To a stirred solution of the corresponding commercially available phenolic compound (1.00 mmol) in acetone is added potassium carbonate (1.50 mmol) and benzyl bromide (1.10 mmol). The solution was then stirred at ambient temperature (˜30° C.) for 16 h. The organic solvent was evaporated under reduced pressure. The residue was diluted with water and extracted with ethyl acetate. The organic layer was dried over anhydrous Na₂SO₄ and evaporated under reduced pressure. The crude compound was purified by column chromatography (ethyl acetate/hexane) to afford corresponding benzylated compound in 80-95% yields.

### Example 7

### Preparation of a Compound of Formula (9′)

### Reaction scheme of Example 7

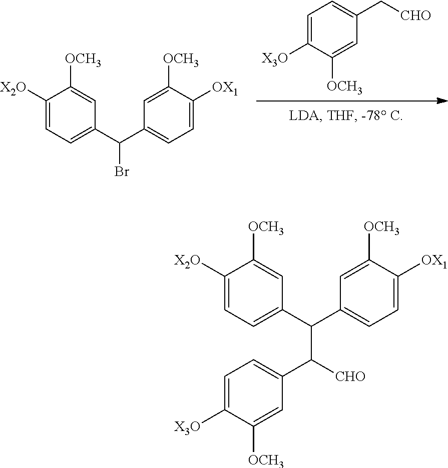

To an oven dried 50 mL RB flask, was added lithium diisopropylamine (3.85 mmol) and THF (10 mL) under N₂ and then was cooled to 0° C. n-BuLi (3.85 mmol, 1.6 M solution in hexane) was added slowly to the above solution under N₂ atmosphere. The solution was then stirred for 30 min at the same temperature. The solution was cooled to −78° C. Compound (7′) in THF (5 mL) was slowly added to the reaction mixture. The stirring was continued for 15 min at the same temperature. Then, freshly prepared brominated compound (6′) (0.77 mmol) in THF (5 mL) was added to the reaction mixture and the solution was stirred at the same temperature for 30 min. TLC analysis indicated complete conversion of compound (6′). The reaction mixture was allowed to reach 0° C., quenched with cold water, and extracted into ethyl acetate. The organic layer was dried over anhydrous Na₂SO₄ and evaporated under reduced pressure. The crude compound was purified by column chromatography (ethyl acetate:hexane 30/70 v/v) to afford compound (9′).

### Example 8

### Preparation of a Compound of Formula (10′) Quebecol

### Reaction Scheme of Example 8

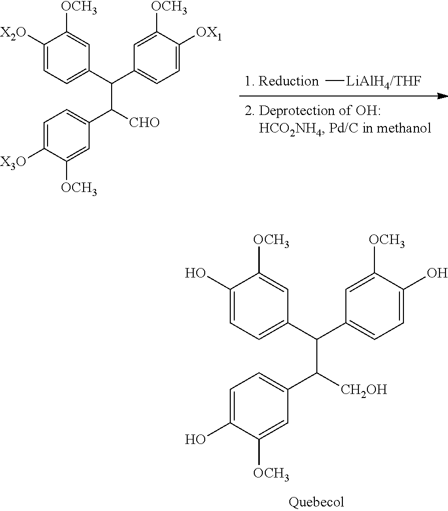

To an oven dried 10 mL RB flask, was added lithium aluminum hydride (0.26 mmol) under N₂ atmosphere. The flask was cooled to 0° C. THF (2 mL) was slowly added followed by compound (9′) in THF (2 mL) to the flask. The solution was stirred at ambient temperature (˜30° C.) for 1 h. TLC analysis indicated complete conversion of compound (9′). The reaction mixture was quenched with saturated NH₄Cl solution and extracted with ethyl acetate. The organic layer was dried over anhydrous Na₂SO₄ and evaporated the volatiles. The crude compound was purified by column chromatography (ethyl acetate:hexane 50/50 v/v). To a stirred solution of the crude compound in methanol was added ammonium formate and Pd/C. The reaction mixture was stirred for 16 h at ambient temperature (˜30° C.). TLC analysis indicated complete conversion of the crude compound. The reaction mixture was filtered through celite pad and the bed was washed with ethyl acetate. The filtrate was evaporated to dryness under reduced pressure. The crude compound was purified by column chromatography (ethyl acetate:hexane 70:30 v/v) to produce Quebecol (10′) as off-white solid (yield 67%). These reactions will be conducted under conditions that will be optimized for maximum yield of product.

### Example 9

### Alternative Synthesis of Quebecol

FIG. 3 illustrates the general procedure for the synthesis of Quebecol. Bis(4-(benzyloxy)-3-methoxyphenyl)methanol (3) is synthesized from the reaction of 1-(benzyloxy)-4-bromo-2-methoxybenzene (1) with 4-(benzyloxy)-3-methoxybenzaldehyde (2) in the presence of n-butyllithium in THF. Bromination of compound 3 in the presence of acetyl bromide in benzene generated the crude building block 4,4′-(bromomethylene)bis(1-(benzyloxy)-2-methoxybenzene) (4) that is used immediately for the reaction with ethyl 2-(4-(benzyloxy)-3-methoxyphenyl)acetate (5) in the presence of LDA to afford tribenzylated compound 6. Subsequent reduction of ethyl ester to alcohol 7 in the presence of lithium aluminum hydride followed by debenzylation with ammonium formate and Pd/C afforded Quebecol (8).

### Experimental Procedures

### Generalized Procedure for the Synthesis of Compounds 1-(benzyloxy)-4-bromo-2-methoxybenzene (1), 4-(benzyloxy)-3-methoxybenzaldehyde (2), and ethyl 2-(4-(benzyloxy)-3-methoxyphenyl)acetate (5)

To a stirred solution of the corresponding commercially available phenol (1.00 mmol) in acetone is added potassium carbonate (1.50 mmol) and benzyl bromide (1.10 mmol). The solution is then stirred at ambient temperature (˜30° C.) for 16 h. The organic solvent is evaporated under reduced pressure. The residue is diluted with water and extracted with ethyl acetate. The organic layer is dried over anhydrous Na₂SO₄ and evaporated under reduced pressure. The crude compound is purified by column chromatography (ethyl acetate/hexane) to afford corresponding benzylated compound in 80-95% yields.

### Synthesis of Bis(4-(benzyloxy)-3-methoxyphenyl)methanol (3)

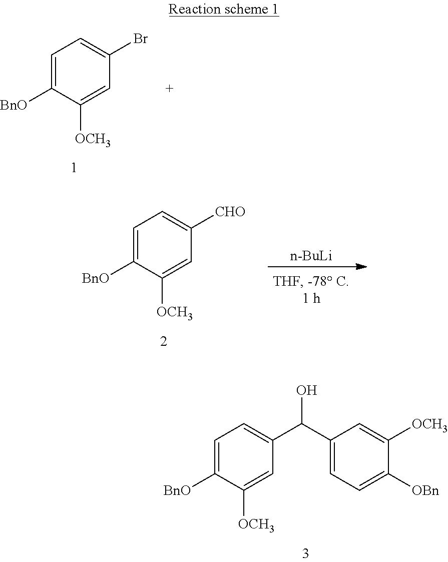

To a stirred solution of 1-(benzyloxy)-4-bromo-2-methoxybenzene (1) (10.23 mmol) in THF (25 mL) at −78° C. slowly is added n-butyllithium (n-BuLi, 10.74 mmol, 1.6 M solution in hexane) under N₂. The mixture is stirred for 30 min at the same temperature. 4-(Benzyloxy)-3-methoxybenzaldehyde (2, 11.26 mmol) in TI-IF (25 mL) is slowly added to the solution over a period of 5 min. Then the solution is stirred for 30 min at −78° C. TLC indicated complete conversion of 1 to the product. The reaction mixture is allowed to reach 0° C., quenched with saturated NH₄Cl solution, and extracted with ethyl acetate. The organic layer is dried over anhydrous Na₂SO₄ and evaporated under reduced pressure. The crude compound is purified by column chromatography (ethyl acetate:hexane 35/65 v/v) to afford 3 as a white solid (yield 55%). FIGS. 4A and B show ¹H NMR spectrum and MS spectrum for compound 3, respectively.

### Synthesis of bis(4-(benzyloxy)-3-methoxyphenyl)bromomethane (4)

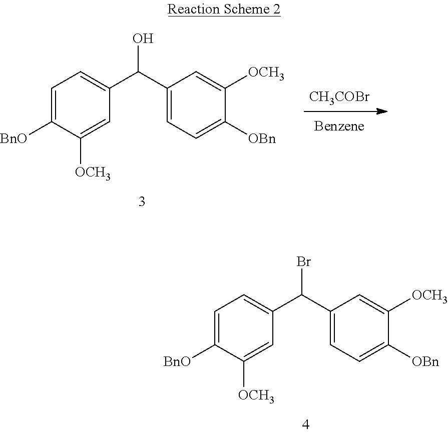

To a slurry of compound 3 (4.82 mmol) in benzene (30 mL) is added acetyl bromide (14.47 mmol) at ambient temperature (˜30° C.) under N₂. The solution is stirred for 5 h. After completion of the reaction, the solvent is evaporated, and the residue is azeotroped with toluene (2 times). The crude compound is washed with hexane (2 times) to remove traces of acetic acid and then dried to yield brominated compound 4 as light pink colour sticky solid, which is used for the next step without further purification (yield 56%).

### Synthesis of Ethyl 2,3,3-tris(4-(benzyloxy)-3-methoxyphenyl)propanoate (6)

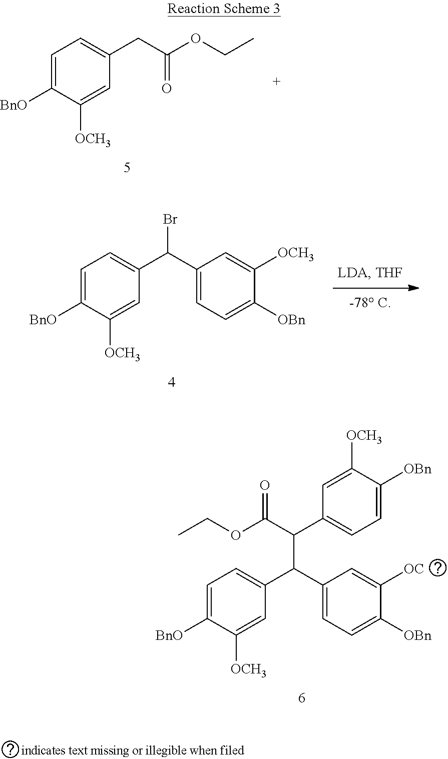

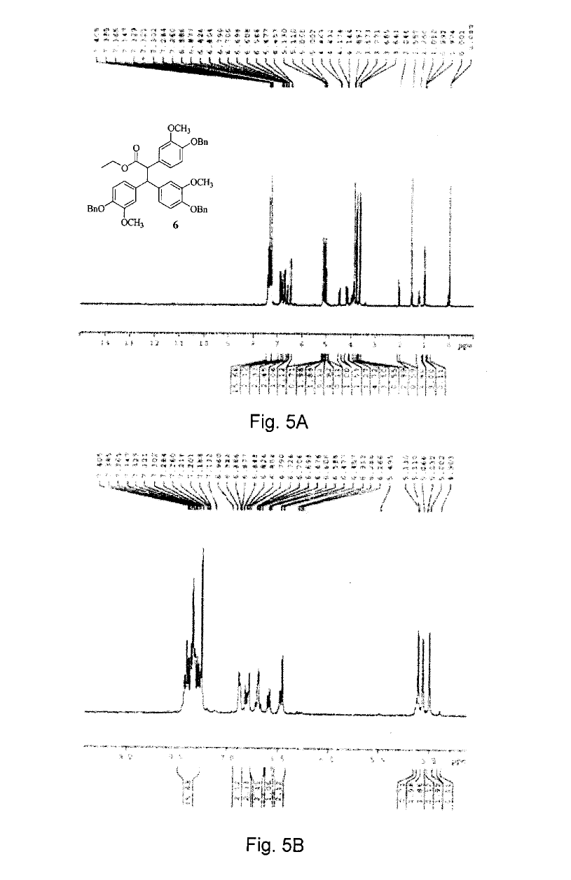

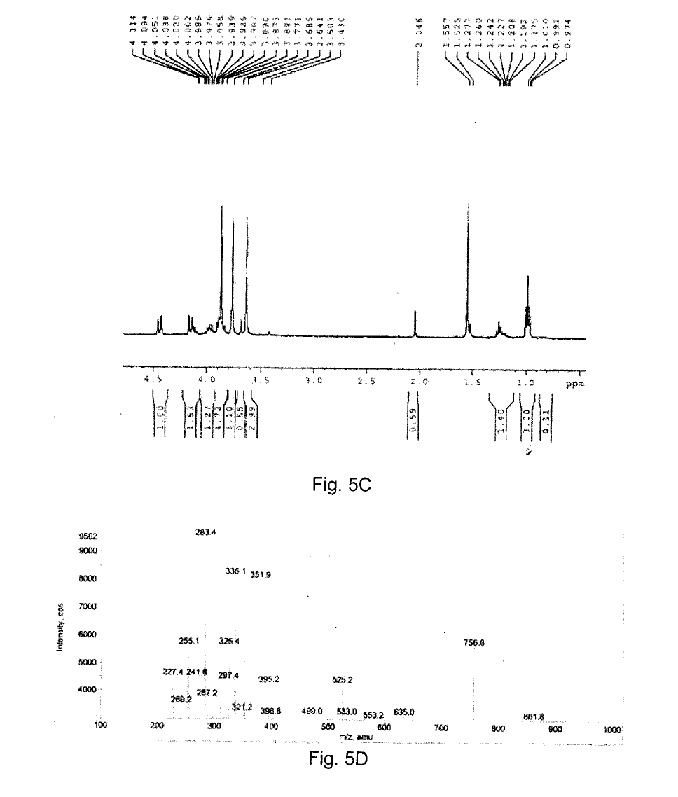

To an oven dried 50 mL RB flask, is added diisopropylamine (3.85 mmol) and THF (10 mL) under N₂ and then is cooled to 0° C. n-BuLi (3.85 mmol, 1.6 M solution in hexane) is added slowly to the above solution under N₂ atmosphere. The solution is then stirred for 30 min at the same temperature. The solution is cooled to −78° C. Ethyl 2-(4-(benzyloxy)-3-methoxyphenyl)acetate 5 (3.08 mmol) in THF (5 mL) is slowly added to the reaction mixture. The stirring is continued for 15 min at the same temperature. Then, freshly prepared brominated compound 4 (0.77 mmol) in THF (5 mL) is added to the reaction mixture and the solution is stirred at the same temperature for 30 min. TLC analysis indicated complete conversion of compound 4. The reaction mixture is allowed to reach 0° C., quenched with cold water, and extracted into ethyl acetate. The organic layer is dried over anhydrous Na₂SO₄ and evaporated under reduced pressure. The crude compound is purified by column chromatography (ethyl acetate:hexane 30/70 v/v) to afford 6 as pale yellow liquid (yield 30%). FIGS. 5A to C show ¹H NMR spectrum and FIG. 5D shows MS spectrum for compound 6.

### Synthesis of 2,3,3-Tris(4-(benzyloxy)-3-methoxyphenyl)propan-1-ol (7)

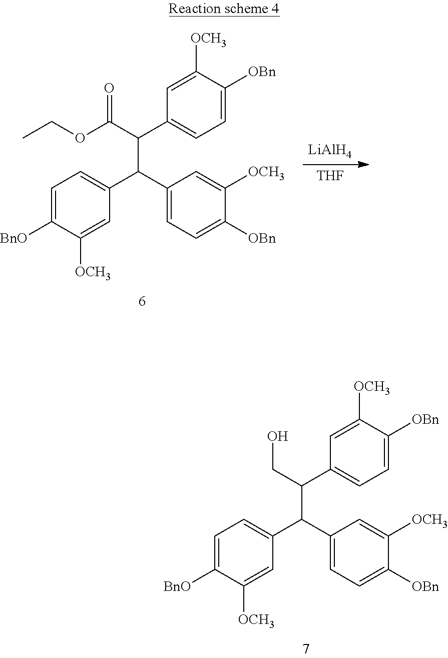 [SMILES: CCOC(=O)C(c1ccc(OCc2ccccc2)c(OC)c1)C(c1ccc(OCc2ccccc2)c(OC)c1)c1ccc(OCc2ccccc2)c(OC)c1 ; "LiAlH4" ; "THF" ; COc1cc(C(CO)C(c2ccc(OCc3ccccc3)c(OC)c2)c2ccc(OCc3ccccc3)c(OC)c2)ccc1OCc1ccccc1]

To an oven dried 10 mL RB flask, is added lithium aluminum hydride (0.26 mmol) under N₂ atmosphere. The flask is cooled to 0° C. THF (2 mL) is slowly added followed by ester 6 in THF (2 mL) to the flask. The solution is stirred at ambient temperature (˜30° C.) for 1 h. TLC analysis indicated complete conversion of ester 6. The reaction mixture is quenched with saturated NH₄Cl solution and extracted with ethyl acetate. The organic layer is dried over anhydrous Na₂SO₄ and evaporated the volatiles. The crude compound is purified by column chromatography (ethyl acetate:hexane 50/50 v/v) to yield 7 as a colorless liquid (yield 76%). FIGS. 6A to C show ¹H NMR spectrum and FIG. 6D shows MS spectrum for compound 7.

### Synthesis of 4,4′,4″-(3-Hydroxypropane-1,1,2-triyl)tris(2-methoxyphenol) (8, Quebecol)

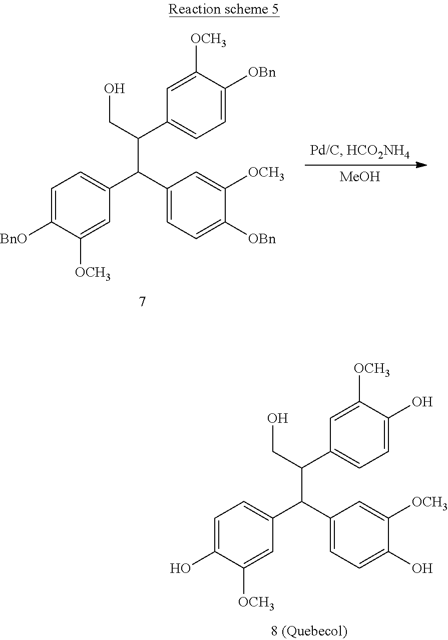 [SMILES: "MeOH" ; COc1cc(C(CO)C(c2ccc(OCc3ccccc3)c(OC)c2)c2ccc(OCc3ccccc3)c(OC)c2)ccc1OCc1ccccc1 ; "Pd/C, HCO2NH4" ; COc1cc(C(CO)C(c2ccc(O)c(OC)c2)c2ccc(O)c(OC)c2)ccc1O]

To a stirred solution of 7 in methanol is added ammonium formate and Pd/C. The reaction mixture is stirred for 16 h at ambient temperature (˜30° C.). TLC analysis indicated complete conversion of 7. The reaction mixture is filtered through celite pad and the bed is washed with ethyl acetate. The filtrate is evaporated to dryness under reduced pressure. The crude compound is purified by column chromatography (ethyl acetate:hexane 70:30 v/v) to produce Quebecol (8) as off-white solid (yield 67%).

### Example 10

### Cytotoxicy of Quebecol and 19 Quebecol Analogs Against Breast Cancer Cells

MTS salt [3-(4,5-dimethylthiazol-2-yl)-5-(3-carboxymethoxyphenyl)-2-(4-sulfenyl)-2H-tetrazolium salt] and etoposide standard are obtained from Sigma-Aldrich. Quebecol is previously isolated in our laboratory as reported (Li and Seeram, 2011) and several analogs are synthesized (see FIGS. 9A and B for codes and structures of the compounds).

Human breast cancer cell lines MCF-7 (estrogen receptor (ER) positive) and MDA-MB-231 (ER negative) are obtained from American Type Culture Collection (Rockville, USA). MCF-7 cells are grown in EMEM medium supplemented with 10% v/v fetal bovine serum, 2% v/v HEPES, 1% v/v nonessential amino acids, 1% v/v L-glutamine and 1% v/v antibiotic solution (Sigma). MDA-MB-231 cells are grown in EMEM medium supplemented with 10% v/v fetal bovine serum, and 1% v/v antibiotic solution. Cells are maintained at 37° C. in an incubator under a 5% CO₂/95% air atmosphere at constant humidity. The pH of the culture medium is determined using pH indicator paper (pHydrion™ Brilliant, pH 5.5-9.0, Micro Essential Laboratory, NY, USA) inside the incubator. Cells are counted using a hemacytometer and are plated at 5000 cells per well, in a 96-well format for 24 h prior to compounds addition. All of the test samples are solubilized in DMSO (<0.5% in the culture medium) and are filter sterilized (0.2 μm) prior to addition to the culture media. Control cells are also run in parallel and subjected to the same changes in medium with a 0.5% DMSO. In addition, cells are treated as indicated above for 24, 48 or 72 h.

At the end of each day of treatment with serially diluted test samples (ranging from 1-200 μg/mL concentrations), 20 μL of the MTS reagent, in combination with the electron coupling agent, phenazine methosulfate, is added to the wells and cells are incubated at 37° C. in a humidified incubator for 3 h. Absorbance at 490 nm (OD₄₉₀) is monitored with a spectrophotometer (SpectraMax M2, Molecular Devices Corp., operated by SoftmaxPro v.4.6 software, Sunnyvale, Calif., USA), to obtain the number of cells relative to control populations. The results are expressed as the concentration that inhibit growth of cell by 50% vs. control cells (control medium used as negative control), IC₅₀. Data are presented as the mean±S.D. of three separated experiments on each cell line (n=2 plates per experiment; 2 wells per treatment per time point). Tamoxifen is used as positive control and provided consistent IC₅₀ values of 16.4±1.1 μg/mL for MCF-7 cells and 10.0±1.4 μg/mL for MDA-MB 231 cells at 72 h of treatment.

Quebecol and its analogs are evaluated for antiproliferative activity in both concentration (ranging from 1-200 μg/mL) and time (at 24, 48, 72, and 96 h) dependent manners by MTS assay. Overall, a clear dose-antiproliferative response is observed in most of compounds. The attached Tables 2 and 3 show the IC₅₀ values of all compounds on breast cancer cell lines at different times (Tables 3A and B: concentrations in μg/mL and Tables 4A and B: concentrations in μM). Most of analogs inhibited proliferation of MCF-7 and MDA-MB 231 cell lines compared to the control cells (0.5% DMSO) in time-dependent manner suggesting that these analogs may have a potential as chemopreventive and chemotherapeutic agents on breast cancer. It should be noted that both the compounds showed similar effects to both MCF-7 and MDA-MB 231 cells.

As shown in Table 2, TRD8 and TRD7 exhibited the highest antiproliferative activities with IC₅₀ values ranging from 10.6-24.8 μg/mL against MCF-7 cells and 17.47-24.0 μg/mL against MDA-MB 231 cells after 72 h of treatment, respectively. These analogs showed better activity on cancer cell lines when compared to Quebecol (46.3±2.1 and 50.7±2.4 μg/mL against the MCF-7 and MDA-MB 231 cells, respectively). Moreover, these two analogs showed IC₅₀ values similar to Tamoxifen used as positive control (Table 2).

Moderate activity, close to the values of Quebecol is showed by other analogs such as QB46, TRD6, QB12, TRD5, and TRD10 with IC₅₀ values ranging from 44.8-78.9 and 62.9-77.4 μg/mL against the MCF-7 and MDA-MB 231 cells, respectively (Table 2).

Finally, analogs such as TRD1, TRD9, QB57, and QB56 showed slight cytotoxicty with IC₅₀ values >80 μg/mL.

TABLE 2
Cytotoxic effects of Quebecol & Quebecol analogs against human
breast cancer cell lines after 72 h treatment.

| Compounds | MCF-7 | MDA-MB-231 |
|---|---|---|
| (g/mL) | IC₅₀ ^{a} | IC₅₀ ^{a} |
| Tamoxifen | 16.4 ± 1.1 | 10.0 ± 1.4 |
| TRD1 | 84.1 ± 3.4 | 93.9 ± 2.4 |
| TRD2 | n.d. | n.d. |
| TRD3 | n.d. | n.d. |
| TRD4 | n.d. | n.d. |
| TRD5 | 75.9 ± 1.9 | 81.6 ± 2.6 |
| TRD6 | 60.4 ± 1.9 | 75.2 ± 1.8 |
| TRD7 | 24.8 ± 1.5 | 24.0 ± 2.1 |
| TRD8 | 10.6 ± 2.1 | 17.4 ± 1.7 |
| TRD9 | 91.4 ± 3.4 | 97.8 ± 3.5 |
| TRD10 | 73.5 ± 1.8 | 77.4 ± 2.7 |
| TRD11 | n.d. | n.d. |
| QB0 | n.d. | n.d. |
| QB12 | 65.0 ± 1.4 | 67.2 ± 1.6 |
| QB39 | n.d. | n.d. |
| QB46 | 44.8 ± 1.7 | 62.9 ± 2.1 |
| QB48 | n.d. | n.d. |
| QB49 | n.d. | n.d. |
| QB56 | 138.1 ± 3.1 | 136.0 ± 3.7 |
| QB57 | n.d. | 85.1 ± 1.8 |
| QUEBECOL | 46.3 ± 2.1 | 50.7 ± 2.4 |
| Natural | | |
| QUEBECOL | 44.4 ± 1.8 | 48.5 ± 1.9 |
| Synthetic | | |

^{a} IC₅₀ (in μg/mL) is defined as the concentration required to achieve 50% inhibition over control cells (DMSO 0.5%); IC₅₀ values are shown as mean ± S.D. from three independent experiments; n.d. = not detected.

TABLE 3A
IC₅₀ concentrations in μg/mL

| | MCF-7 | | MCF-7 | | MCF-7 | | MCF-7 | |
|---|---|---|---|---|---|---|---|---|
| Code | 24 h | SD | 48 h | SD | 72 h | SD | 96 h | SD |
| Tamoxifen | 24.6 | 1.4 | 17.9 | 1.5 | 16.4 | 1.1 | 14.2 | 1.3 |
| TRD1 | 94.2 | 2.6 | 89.1 | 2.3 | 84.1 | 3.4 | 78.3 | 1.9 |
| TRD2 | n.d. | n.d. | n.d. | n.d. | n.d. | n.d. | n.d. | n.d. |
| TRD3 | n.d. | n.d. | n.d. | n.d. | n.d. | n.d. | n.d. | n.d. |
| TRD4 | n.d. | n.d. | n.d. | n.d. | n.d. | n.d. | n.d. | n.d. |
| TRD5 | n.d. | n.d. | 80.9 | 2.0 | 75.9 | 1.9 | 66.5 | 2.3 |
| TRD6 | 78.7 | 1.9 | 66.1 | 2.7 | 60.4 | 1.9 | 43.6 | 2.7 |
| TRD7 | 42.3 | 1.9 | 32.7 | 1.7 | 24.8 | 1.5 | 23.2 | 1.9 |
| TRD8 | 42.0 | 1.5 | 21.8 | 1.7 | 10.6 | 2.1 | 7.0 | 1.9 |
| TRD9 | 110.7 | 4.3 | 102.5 | 2.0 | 91.4 | 3.4 | 82.3 | 2.2 |
| TRD10 | 87.0 | 2.4 | 81.9 | 2.4 | 73.5 | 1.8 | 54.4 | 2.5 |
| TRD11 | n.d. | n.d. | n.d. | n.d. | n.d. | n.d. | n.d. | n.d. |
| QB0 | n.d. | n.d. | n.d. | n.d. | n.d. | n.d. | n.d. | n.d. |
| QB12 | n.d. | n.d. | n.d. | n.d. | 65.0 | 1.4 | 59.6 | 1.3 |
| QB39 | n.d. | n.d. | n.d. | n.d. | n.d. | n.d. | n.d. | n.d. |
| QB46 | 73.2 | 2.7 | 59.5 | 1.9 | 44.8 | 1.7 | 36.8 | 1.9 |
| QB48 | n.d. | n.d. | n.d. | n.d. | n.d. | n.d. | n.d. | n.d. |
| QB49 | n.d. | n.d. | n.d. | n.d. | n.d. | n.d. | n.d. | n.d. |
| QUEBECOL | 65.9 | 2.0 | 53.5 | 2.2 | 46.3 | 2.1 | 37.4 | 1.7 |
| QB56 | n.d. | n.d. | n.d. | n.d. | 138.1 | 3.1 | 124.9 | 3.8 |
| QB57 | n.d. | n.d. | n.d. | n.d. | n.d. | n.d. | n.d. | n.d. |
| QUEBECOL | 67.2 | 2.2 | 54.2 | 1.8 | 44.4 | 1.8 | 38.0 | 1.9 |
| SYNTHETIC | | | | | | | | |

IC₅₀ (in μg/mL) is defined as the concentration required to achieve 50% inhibition over control cells (DMSO 0.5%); IC₅₀ values are shown as mean ± S.D. from three independent experiments;
n.d. = not detected.

TABLE 3B
IC₅₀ concentrations in μg/mL

| | MDA-MB- | | MDA-MB- | | MDA-MB- | | MDA-MB- | |
|---|---|---|---|---|---|---|---|---|
| | 231 | | 231 | | 231 | | 231 | |
| Code | 24 h | SD | 48 h | SD | 72 h | SD | 96 h | SD |
| Tamoxifen | 26.0 | 1.0 | 19.7 | 1.5 | 10.0 | 1.4 | 7.1 | 1.5 |
| TRD1 | n.d. | n.d. | n.d. | n.d. | 93.9 | 2.4 | 89.4 | 2.3 |
| TRD2 | n.d. | n.d. | n.d. | n.d. | n.d. | n.d. | n.d. | n.d. |
| TRD3 | n.d. | n.d. | n.d. | n.d. | n.d. | n.d. | n.d. | n.d. |
| TRD4 | n.d. | n.d. | n.d. | n.d. | n.d. | n.d. | n.d. | n.d. |
| TRD5 | n.d. | n.d. | n.d. | n.d. | 81.6 | 2.6 | 78.2 | 2.2 |
| TRD6 | n.d. | n.d. | n.d. | n.d. | 75.2 | 1.8 | 71.5 | 1.6 |
| TRD7 | 36.4 | 1.6 | 30.4 | 1.9 | 24.0 | 2.1 | 15.6 | 1.9 |
| TRD8 | 43.5 | 2.0 | 27.8 | 1.8 | 17.4 | 1.7 | 11.2 | 2.0 |
| TRD9 | 114.9 | 2.3 | 107.5 | 2.5 | 97.8 | 3.5 | 89.9 | 2.5 |
| TRD10 | n.d. | n.d. | 85.8 | 2.1 | 77.4 | 2.7 | 68.7 | 2.0 |
| TRD11 | n.d. | n.d. | n.d. | n.d. | n.d. | n.d. | n.d. | n.d. |
| QB0 | n.d. | n.d. | n.d. | n.d. | n.d. | n.d. | 24.0 | 0.7 |
| QB12 | n.d. | n.d. | n.d. | n.d. | 67.2 | 1.6 | 58.0 | 2.1 |
| QB39 | n.d. | n.d. | n.d. | n.d. | n.d. | n.d. | n.d. | n.d. |
| QB46 | 78.3 | 1.3 | 72.4 | 2.8 | 62.9 | 2.1 | 53.0 | 1.7 |
| QB48 | n.d. | n.d. | n.d. | n.d. | n.d. | n.d. | 51.2 | 1.7 |
| QB49 | n.d. | n.d. | n.d. | n.d. | n.d. | n.d. | n.d. | n.d. |
| QUEBECOL | 70.9 | 2.3 | 57.0 | 1.8 | 50.7 | 2.4 | 43.3 | 1.6 |
| QB56 | n.d. | n.d. | 143.3 | 2.8 | 136.0 | 3.7 | 129.3 | 3.9 |
| QB57 | n.d. | n.d. | n.d. | n.d. | 85.1 | 1.8 | 73.8 | 2.9 |
| QUEBECOL | 72.5 | 2.0 | 55.6 | 2.2 | 48.5 | 1.9 | 43.8 | 1.7 |
| SYNTHETIC | | | | | | | | |

IC₅₀ (in μg/mL) is defined as the concentration required to achieve 50% inhibition over control cells (DMSO 0.5%); IC₅₀ values are shown as mean ± S.D. from three independent experiments;
n.d. = not detected.

TABLE 4A
IC₅₀ concentrations in μM

| | MCF-7 | | MCF-7 | | MCF-7 | | MCF-7 | |
|---|---|---|---|---|---|---|---|---|
| Code | 24 h | SD | 48 h | SD | 72 h | SD | 96 h | SD |
| Tamoxifen | 66.2 | 3.8 | 48.3 | 4.1 | 44.1 | 2.9 | 38.2 | 3.5 |
| TRD1 | 195.2 | 5.3 | 184.7 | 4.8 | 174.3 | 7.1 | 162.4 | 3.9 |
| TRD2 | n.d. | n.d. | n.d. | n.d. | n.d. | n.d. | n.d. | n.d. |
| TRD3 | n.d. | n.d. | n.d. | n.d. | n.d. | n.d. | n.d. | n.d. |
| TRD4 | n.d. | n.d. | n.d. | n.d. | n.d. | n.d. | n.d. | n.d. |
| TRD5 | n.d. | n.d. | 195.2 | 4.8 | 183 | 4.7 | 160.4 | 5.5 |
| TRD6 | 197.5 | 4.8 | 165.8 | 6.7 | 151.6 | 4.8 | 109.4 | 6.7 |
| TRD7 | 104.6 | 4.7 | 80.9 | 4.3 | 61.3 | 3.8 | 57.3 | 4.6 |
| TRD8 | 96.7 | 3.5 | 50.1 | 3.9 | 24.3 | 4.9 | 16.2 | 4.3 |
| TRD9 | 188.6 | 7.3 | 174.6 | 3.4 | 155.7 | 5.8 | 140.2 | 3.8 |
| TRD10 | 191.7 | 5.3 | 180.4 | 5.2 | 162 | 3.9 | 119.9 | 5.6 |
| TRD11 | n.d. | n.d. | n.d. | n.d. | n.d. | n.d. | n.d. | n.d. |
| QB0 | n.d. | n.d. | n.d. | n.d. | n.d. | n.d. | n.d. | n.d. |
| QB12 | n.d. | n.d. | n.d. | n.d. | 185.4 | 4.1 | 170.2 | 3.7 |
| QB39 | n.d. | n.d. | n.d. | n.d. | n.d. | n.d. | n.d. | n.d. |
| QB46 | 160.4 | 5.9 | 130.4 | 4.2 | 98.1 | 3.7 | 80.7 | 4.1 |
| QB48 | n.d. | n.d. | n.d. | n.d. | n.d. | n.d. | n.d. | n.d. |
| QB49 | n.d. | n.d. | n.d. | n.d. | n.d. | n.d. | n.d. | n.d. |
| QUEBECOL | 154.6 | 4.6 | 125.5 | 5.2 | 108.8 | 4.9 | 87.9 | 3.9 |
| Natural | | | | | | | | |
| QB56 | n.d. | n.d. | n.d. | n.d. | 188.1 | 4.2 | 170.1 | 5.2 |
| QB57 | n.d. | n.d. | n.d. | n.d. | n.d. | n.d. | n.d. | n.d. |
| QUEBECOL | 157.7 | 5.1 | 127.2 | 4.2 | 104.3 | 4.2 | 89.1 | 4.5 |
| SYNTHETIC | | | | | | | | |

IC₅₀ (in μM) is defined as the concentration required to achieve 50% inhibition over control cells (DMSO 0.5%); IC₅₀ values are shown as mean ± S.D. from three independent experiments;
n.d. = not detected.

TABLE 4B
IC₅₀ concentrations in μM

| | MDA-MB- | | MDA-MB- | | MDA-MB- | | MDA-MB- |
|---|---|---|---|---|---|---|---|
| | 231 | | 231 | | 231 | | 231 |
| Code | 24 h | SD | 48 h | SD | 72 h | SD | 96 h |
| Tamoxifen | 70.1 | 2.6 | 53.1 | 4.1 | 26.8 | 3.9 | 19 |
| TRD1 | n.d. | n.d. | n.d. | n.d. | 194.6 | 5 | 185.4 |
| TRD2 | n.d. | n.d. | n.d. | n.d. | n.d. | n.d. | n.d. |
| TRD3 | n.d. | n.d. | n.d. | n.d. | n.d. | n.d. | n.d. |
| TRD4 | n.d. | n.d. | n.d. | n.d. | n.d. | n.d. | n.d. |
| TRD5 | n.d. | n.d. | n.d. | n.d. | 196.8 | 6.2 | 188.7 |
| TRD6 | n.d. | n.d. | n.d. | n.d. | 188.7 | 4.6 | 179.4 |
| TRD7 | 90.0 | 3.9 | 75.3 | 4.8 | 59.4 | 5.1 | 38.7 |
| TRD8 | 100.2 | 4.6 | 63.9 | 4.2 | 40 | 3.8 | 25.8 |
| TRD9 | 195.7 | 3.9 | 183.1 | 4.3 | 166.6 | 6 | 153.2 |
| TRD10 | n.d. | n.d. | 189.2 | 4.6 | 170.5 | 5.9 | 151.3 |
| TRD11 | n.d. | n.d. | n.d. | n.d. | n.d. | n.d. | n.d. |
| QB0 | n.d. | n.d. | n.d. | n.d. | n.d. | n.d. | 193.4 |
| QB12 | n.d. | n.d. | n.d. | n.d. | 191.8 | 4.6 | 165.4 |
| QB39 | n.d. | n.d. | n.d. | n.d. | n.d. | n.d. | n.d. |
| QB46 | 171.5 | 2.9 | 158.6 | 6.1 | 137.8 | 4.6 | 116.2 |
| QB48 | n.d. | n.d. | n.d. | n.d. | n.d. | n.d. | 185.3 |
| QB49 | n.d. | n.d. | n.d. | n.d. | n.d. | n.d. | n.d. |
| QUEBECOL | 166.4 | 5.3 | 133.8 | 4.2 | 119.1 | 5.7 | 101.6 |
| Natural | | | | | | | |
| QB56 | n.d. | n.d. | 195.2 | 3.8 | 185.3 | 5 | 176.1 |
| QB57 | n.d. | n.d. | n.d. | n.d. | 181.9 | 3.9 | 157.6 |
| QUEBECOL | | | | | | | |
| SYNTHETIC | | | | | | | |
| | 170.1 | 4.7 | 130.4 | 5.1 | 113.9 | 4.5 | 102.8 |

IC₅₀ (in μM) is defined as the concentration required to achieve 50% inhibition over control cells (DMSO 0.5%); IC₅₀ values are shown as mean ± S.D. from three independent experiments;
n.d. = not detected.

TABLE 5A

| | MCF-7 | | MCF-7 | | MCF-7 | | MCF-7 | |
|---|---|---|---|---|---|---|---|---|
| Code | 24 h | SD | 48 h | SD | 72 h | SD | 96 h | SD |
| Tamoxifen | 66.2 | 3.8 | 48.3 | 4.1 | 44.1 | 2.9 | 38.2 | 3.5 |
| QUEBECOL | 154.6 | 4.6 | 125.5 | 5.2 | 108.8 | 4.9 | 87.9 | 3.9 |
| T + Q (1:1) | 141.5 | 3.8 | 123.4 | 2.9 | 108.1 | 3.8 | 77.1 | 4.7 |

TABLE 5B

| | MDA-MB- | | MDA-MB- | | MDA-MB- | | MDA-MB- | |
|---|---|---|---|---|---|---|---|---|
| | 231 | | 231 | | 231 | | 231 | |
| Code | 24 h | SD | 48 h | SD | 72 h | SD | 96 h | SD |
| Tamoxifen | 70.1 | 2.6 | 53.1 | 4.1 | 26.8 | 3.9 | 19 | 4.1 |
| QUEBECOL | 166.4 | 5.3 | 133.8 | 4.2 | 119.1 | 5.7 | 101.6 | 3.7 |
| T + Q (1:1) | 152.9 | 4.2 | 125.7 | 2.7 | 105.3 | 4.1 | 78.1 | 3.2 |

IC₅₀ (in μM) is defined as the concentration required to achieve 50% inhibition over control cells (DMSO 0.5%); IC₅₀ values are shown as mean ± S.D. from three independent experiments;
n.d. = not detected.
T (starting concentration with 50 μM) and Q (start concentration with 200 μM).

As a last part of this example, the possible synergistic effects of Quebecol and tamoxifen are evaluated. Tables 5 A and B shows the IC₅₀ values of these compounds of the combination (1:1) of both compounds. The data did not show any significant enhanced effects of the combination when compared to the compounds alone.

Cancer is a leading cause of death worldwide. The current study investigated the in vitro anticancer activities of a process-derived phenolic compound, Quebecol, present in maple syrup and 19 different analogs. It should be noted that both natural and synthetic Quebecol showed similar activity.

Given that these compounds have never been investigated for their anticancer potential, their cytotoxic effects against breast cancer lines (MCF-7 and MDA-MB 321) is investigated. The compounds are evaluated for both time and concentration dependent effects.

Two analogs, TRD8 and TRD7, exerted the highest antiproliferative activities against MCF-7 cells and MDA-MB 231 cells after 72 h of treatment, respectively. Notably, this cytotoxic activity on both breast cancer cell lines is higher than exerted by Quebecol, and very similar to the activity exerted by Tamoxifen, used as positive control. Other analogs such as QB46, TRD6, QB12, TRD5, and TRD10 showed a moderate cytotoxic activity.

The results indicate, for the first time, that Quebecol and some of its analogs exert cytotoxic effects on breast cancer cell lines in both time and concentration dependent manners, suggesting that they may have potential as cancer chemopreventive and/or chemotherapeutic agents. Quebecol has previously been shown to have cytotoxic effect on colon cancer cell lines (Gonzales-Sarrias et al. F. Func. Food. 4, 1, 185-196, 2011). The highest activity is exerted by two analogs TRD8 and TRD7.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A compound of formula (I): 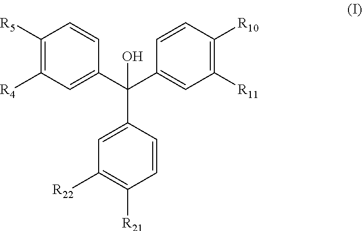
wherein
R₅, R₁₀, and R₂₁ are OCH₃,
R₄, R₁₁, and R₂₂ are independently chosen from OH, Cl, Br, F, CF₃, CH₃ and CHO,
pharmaceutically acceptable salt, racemic mixture, enantiomer, diastereoisomer, isomer, and tautomer thereof.

2. A compound of formula TRD6: 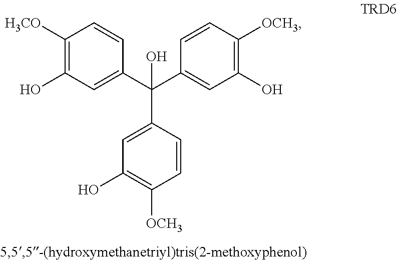 [SMILES: COc1ccc(C(O)(c2ccc(OC)c(O)c2)c2ccc(OC)c(O)c2)cc1O]
pharmaceutically acceptable salt, racemic mixture, enantiomer, diastereoisomer, isomer, and tautomer thereof.

3. A compound of formula TRD8: 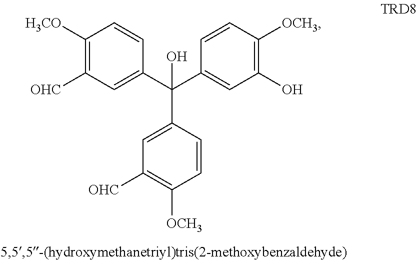 [SMILES: COc1ccc(C(O)(c2ccc(OC)c(C=O)c2)c2ccc(OC)c(C=O)c2)cc1O]
pharmaceutically acceptable salt, racemic mixture, enantiomer, diastereoisomer, isomer, and tautomer thereof.

4. A compound of formula TRD9: 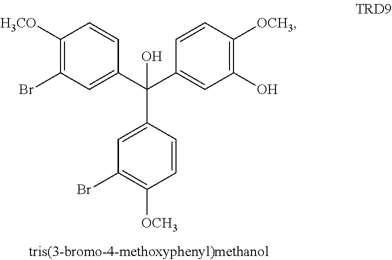 [SMILES: COc1ccc(C(O)(c2ccc(OC)c(Br)c2)c2ccc(OC)c(Br)c2)cc1O]
pharmaceutically acceptable salt, racemic mixture, enantiomer, diastereoisomer, isomer, and tautomer thereof.

5. A compound of formula TRD10: 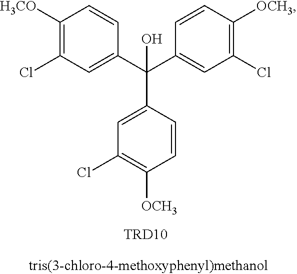
pharmaceutically acceptable salt, racemic mixture, enantiomer, diastereoisomer, isomer, and tautomer thereof.

6. A compound of formula QB12, QB48, QB49, QB56, and QB57: 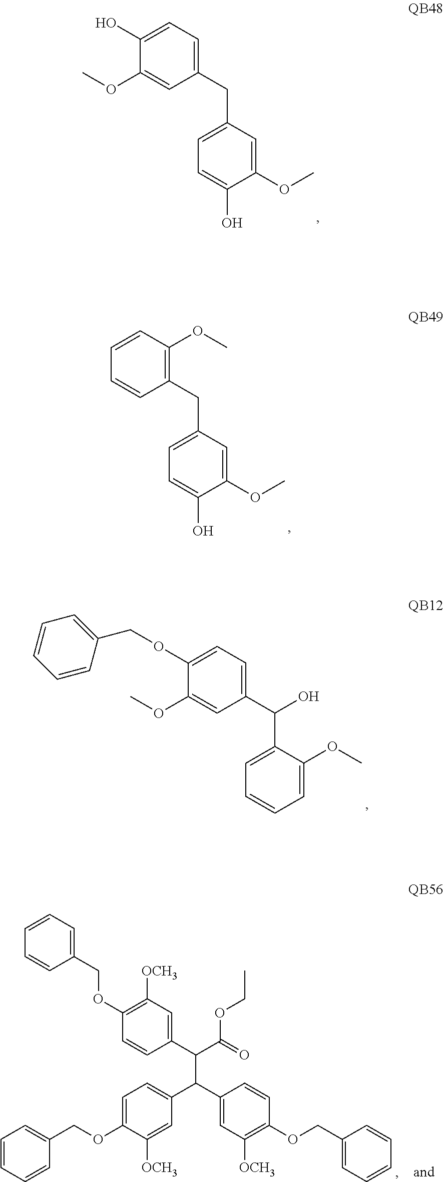 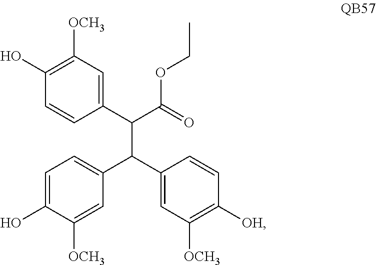 [SMILES: CCOC(=O)C(c1ccc(O)c(OC)c1)C(c1ccc(O)c(OC)c1)c1ccc(O)c(OC)c1]
pharmaceutically acceptable salt, racemic mixture, enantiomer, diastereoisomer, isomer, and tautomer thereof.

7. A pharmaceutical composition comprising a therapeutically effective amount of a compound according to any one of claims 1-6.

8. A method to inhibit tumor growth in a subject, which comprises administering a composition according to claim 7.

9. A method to inhibit tumor growth in a subject, which comprises administering an anticancer amount of a compound chosen from TRD1, TRD5, TRD6, TRD7, TRD8, TRD9, TRD10, QB12, QB39, QB46, QB56, and QB57:

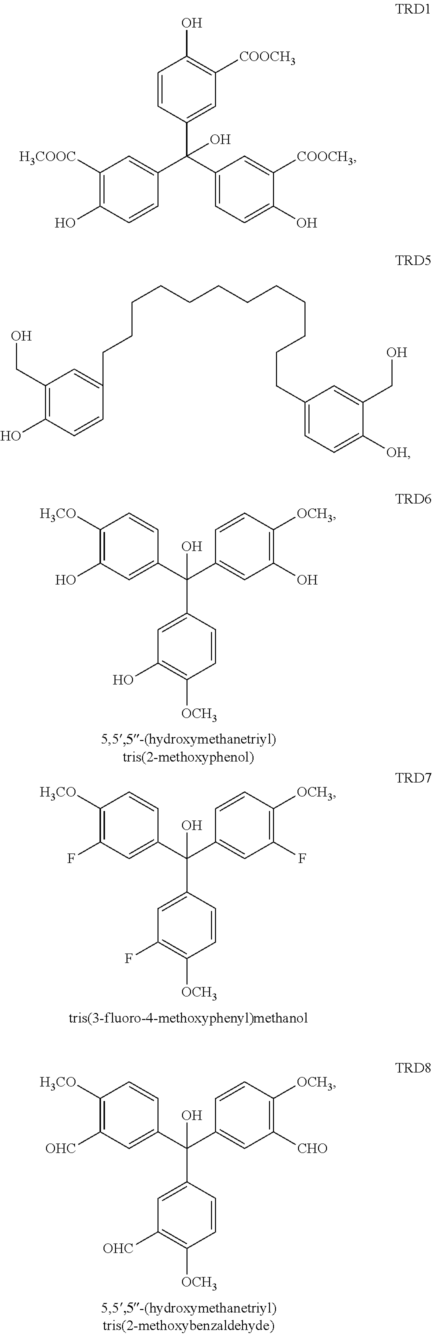

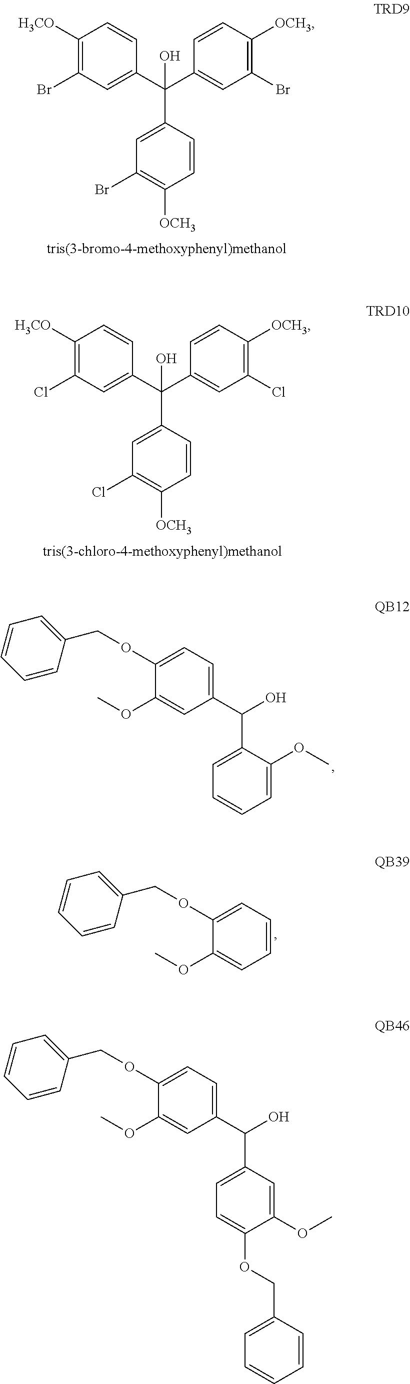 [SMILES: COc1ccc(C(O)(c2ccc(OC)c(Br)c2)c2ccc(OC)c(Br)c2)cc1Br ; COc1ccc(C(O)(c2ccc(OC)c(Cl)c2)c2ccc(OC)c(Cl)c2)cc1Cl ; COc1cc(C(O)c2ccccc2OC)ccc1OCc1ccccc1 ; COc1ccccc1OCc1ccccc1 ; COc1cc(C(O)c2ccc(OCc3ccccc3)c(OC)c2)ccc1OCc1ccccc1]

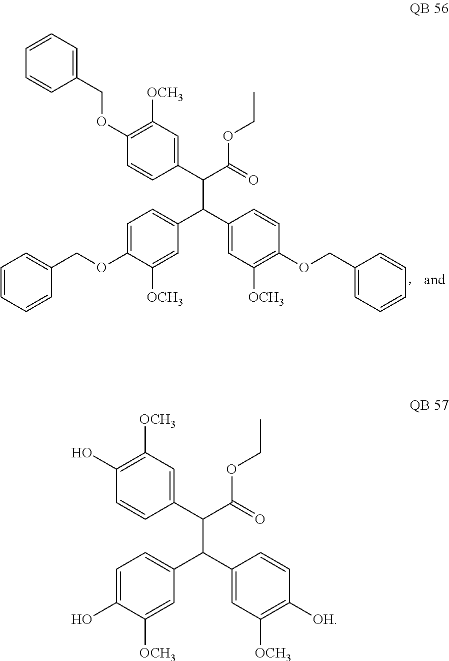 [SMILES: CCOC(=O)C(c1ccc(OCc2ccccc2)c(OC)c1)C(c1ccc(OCc2ccccc2)c(OC)c1)c1ccc(OCc2ccccc2)c(OC)c1 ; CCOC(=O)C(c1ccc(O)c(OC)c1)C(c1ccc(O)c(OC)c1)c1ccc(O)c(OC)c1]

10. Use of a compound as claimed in any one of claims 1-6, for the preparation of a medicament for the treatment of cancer.

11. Use of a compound as claimed in any one of claims 1-6, for the treatment of cancer.

12. Use of a compound chosen from TRD1, TRD5, TRD6, TRD7, TRD8, TRD9, TRD10, QB12, QB39, QB46, QB56, and QB57 to inhibit tumor growth in a subject:

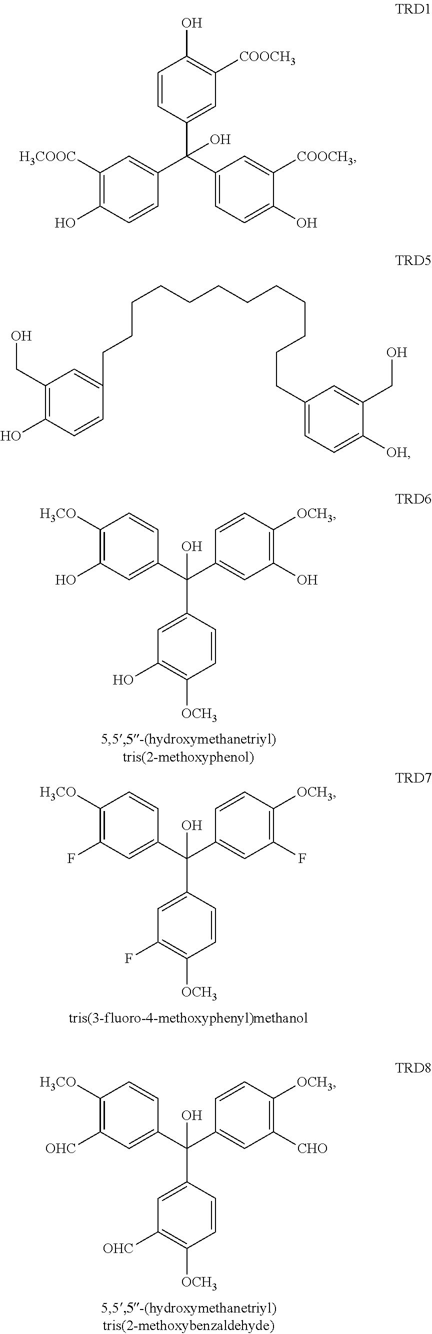

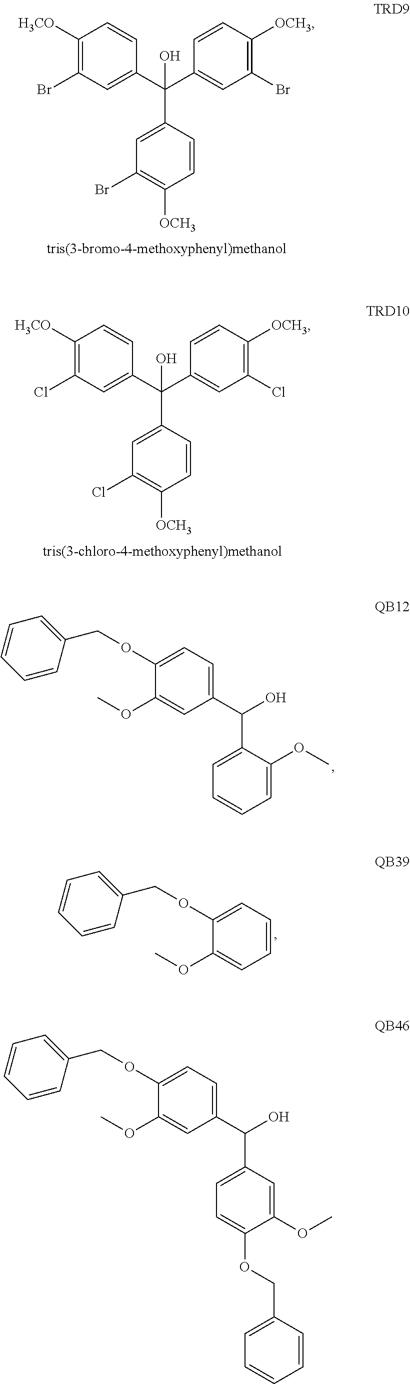 [SMILES: COc1ccc(C(O)(c2ccc(OC)c(Br)c2)c2ccc(OC)c(Br)c2)cc1Br ; COc1ccc(C(O)(c2ccc(OC)c(Cl)c2)c2ccc(OC)c(Cl)c2)cc1Cl ; COc1cc(C(O)c2ccccc2OC)ccc1OCc1ccccc1 ; COc1ccccc1OCc1ccccc1 ; COc1cc(C(O)c2ccc(OCc3ccccc3)c(OC)c2)ccc1OCc1ccccc1]

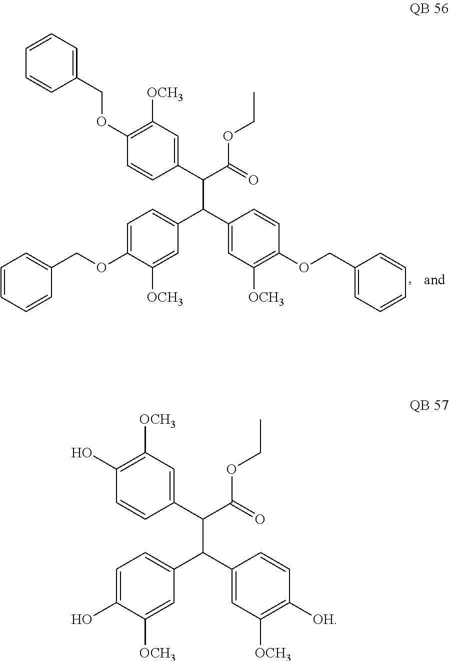 [SMILES: CCOC(=O)C(c1ccc(OCc2ccccc2)c(OC)c1)C(c1ccc(OCc2ccccc2)c(OC)c1)c1ccc(OCc2ccccc2)c(OC)c1 ; CCOC(=O)C(c1ccc(O)c(OC)c1)C(c1ccc(O)c(OC)c1)c1ccc(O)c(OC)c1]

13. The use as claimed in claim 12, wherein said tumor is a breast tumor, a prostate tumor, a lung tumor, a colon tumor, a liver tumor and a testes tumor.

14. A process for the synthesis of a compound of formula (5′): 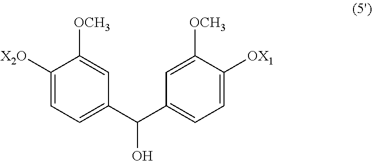
said process comprising the step of:
i. reacting a compound of formula (2′) 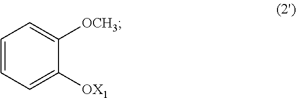
with a compound of formula (4′) 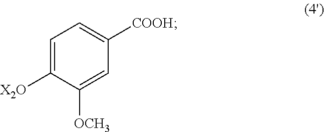
to obtain said compound of formula (5′)
wherein X₁, and X₂ is a suitable protecting group for a hydroxyl group.

15. A process for the synthesis of a compound of formula (6′) 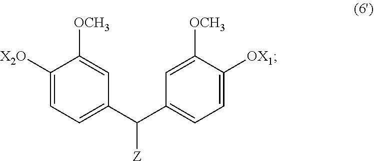
said process comprising the step of:
i. reacting a compound of formula (5′) 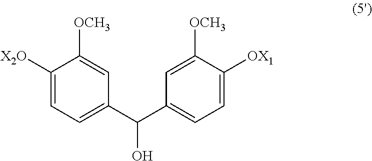
with a halogenating agent to obtain said compound of formula (6′),
wherein X₁, and X₂ is a suitable protecting group for a hydroxyl group, and
wherein Z is a halogen atom.

16. A process for the synthesis of a compound of formula (8′) 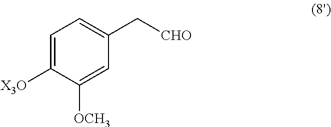
said process comprising the step of:
i. reacting a compound of formula (7′) 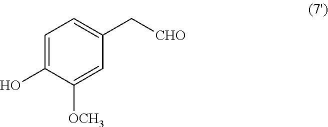 [SMILES: COc1cc(CC=O)ccc1O]
with a suitable hydroxyl protecting group, to obtain said compound of formula (8′)
wherein X₃ is a suitable protecting group for a hydroxyl group; and
wherein Z is a halogen atom.

17. A process for the synthesis of a compound of formula (9′) 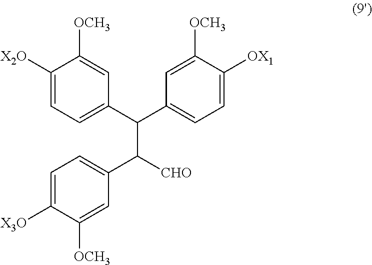
said process comprising the step of:
i. reacting a compound of formula (6′) 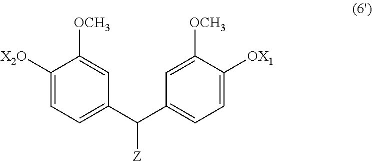
with a compound of formula (8′) 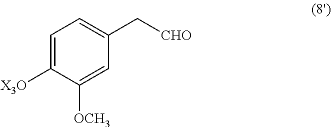
to obtain said compound of formula (9′),
wherein X₁, X₂ and X₃ is a suitable protecting group for a hydroxyl group; and
wherein Z is a halogen atom.

18. A process for the synthesis of a compound of formula (10′) (Quebecol) 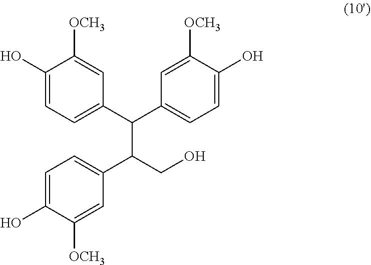 [SMILES: COc1cc(C(CO)C(c2ccc(O)c(OC)c2)c2ccc(O)c(OC)c2)ccc1O]
said process comprising the steps of:
i. reducing and deprotecting a compound of formula (9′) 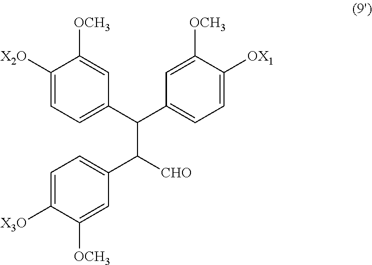
to obtain said compound of formula (10′) (Quebecol);
wherein X₁, X₂ and X₃ is a suitable protecting group for a hydroxyl group; and
wherein Z is a halogen atom.

19. A process for the synthesis of a compound of formula (10′) (Quebecol) 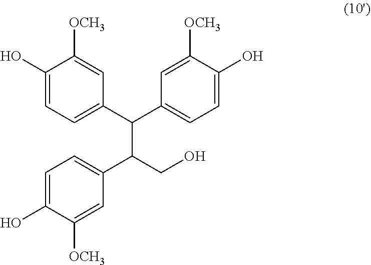 [SMILES: COc1cc(C(CO)C(c2ccc(O)c(OC)c2)c2ccc(O)c(OC)c2)ccc1O]
said process comprising the steps of:
i. reacting a compound of formula (1′) 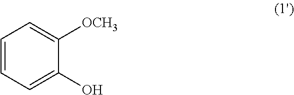
with a suitable hydroxyl protecting group, to obtain a compound of formula (2) 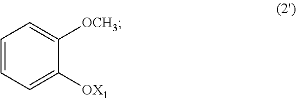
ii. reacting a compound of formula (3′) 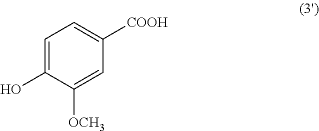 [SMILES: COc1cc(C(=O)O)ccc1O]
with a suitable hydroxyl protecting group, to obtain a compound of formula (4′) 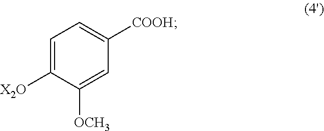
iii. reacting said compound of formula (2′) with said compound of formula (4′) to obtain a compound of formula (5′) 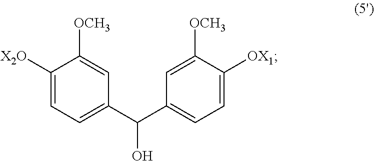
iv. reacting said compound of formula (5′) with an halogenating agent to obtain a compound of formula (6′) 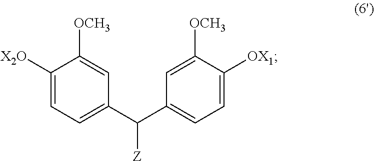
v. reacting a compound of formula (7′) 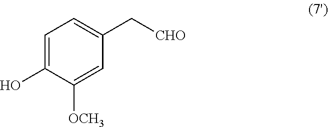 [SMILES: COc1cc(CC=O)ccc1O]
with a suitable hydroxyl protecting group, to obtain a compound of formula (8) 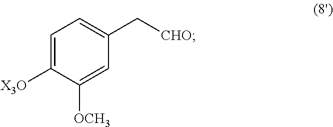
vi. reacting said compound of formula (6′) with said compound of formula (8′) to obtain a compound of formula (9′) 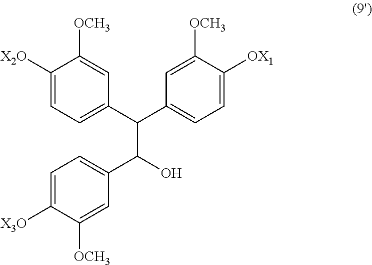
vii. reducing and deprotecting said compound of formula (9′) to obtain a compound of formula (10′) (Quebecol);
wherein X₁, X₂ and X₃ is a suitable protecting group for a hydroxyl group; and
wherein Z is a halogen atom.

20. The process according to any one of claims 16 to 19, wherein said X₃ is chosen from Fluorenylmethyloxycarbonyl chloride (FMOC), Triphenylmethyl chloride, and a silyl ether.

21. The process according to any one of claims 16 to 19, wherein said X₃ is a silyl ether.

22. The process according to any one of claims 18-19, wherein said reducing is by reacting said compound (9′) with NaBH₄.

23. The process according to any one of claims 18-19, wherein said deprotection is by reacting said compound of formula (9′) with one of tetra-n-butylammonium fluoride (TBAF) or trifluoroacetic acid (TFA).

24. The process according to claim 19, wherein in step iv), said halogenating agent is a trihalide of phosphorus.

25. The process according to claim 19, wherein said trihalide of phosphorous is chosen from PBr₃, and PCl_{S}.

26. A process for the synthesis of a compound of formula (3) 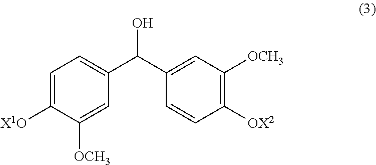
said process comprising the step of:
i. reacting a compound of formula (1) 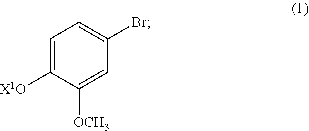
with a compound of formula (2) 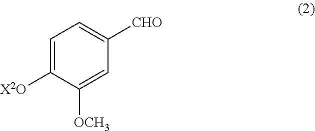
in presence of a strong base, to obtain a compound of formula (3)
wherein X¹ and X² is a suitable protecting group for a hydroxyl group.

27. The process of claim 26, wherein said strong base is n-butyllithium (n-BuLi).

28. The process of claim 27, wherein reacting is in tetrahydrofuran (THF) at −78° C.

29. A process for the synthesis of a compound of formula (4) 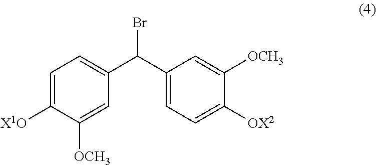
said process comprising the step of:
i. brominating a compound of formula (3) 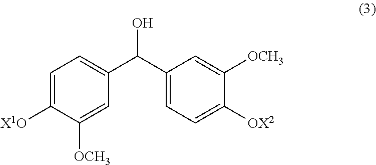
to obtain said compound of formula (4)
wherein X¹ and X² is a suitable protecting group for a hydroxyl group.

30. The process of claim 29, wherein brominating is with acetyl bromide (CH₃COBr).

31. The process of claim 29, wherein brominating is with acetyl bromide (CH₃COBr) in benzene.

32. A process for the synthesis of a compound of formula (6) 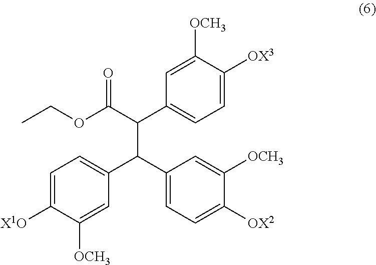
said process comprising the step of:
i. reacting a compound of formula (4) 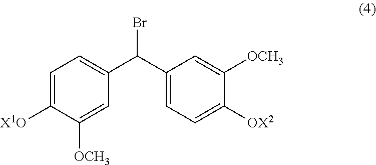
with a compound of formula (5) 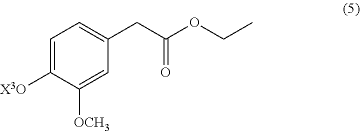
in the presence of a strong base, to obtain said compound of formula (6),
wherein X¹, X², and X³ is a suitable protecting group for a hydroxyl group.

33. The process of claim 32, wherein said strong base is Lithium diisopropylamide (LDA).

34. The process of claim 32, wherein said strong base is Lithium diisopropylamide (LDA) in tetrahydrofuran at tetrahydrofuran (THF) at −78° C.

35. A process for the synthesis of a compound of formula (7) 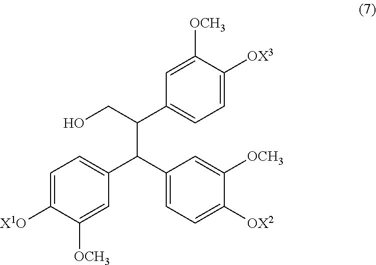
said process comprising the step of:
i. reducing a compound of formula (6) 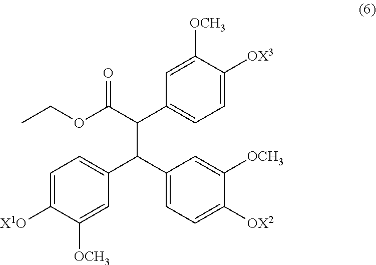
to obtain said compound of formula (7),
wherein X¹, X², and X³ is a suitable protecting group for a hydroxyl group.

36. The process of claim 35, wherein reducing is with lithium aluminum hydride (LiAlH₄).

37. The process of claim 35, wherein reducing is with lithium aluminum hydride (LiAlH₄) in tetrahydrofuran (THF).

38. A process for the synthesis of a compound of formula (8) (Quebecol) 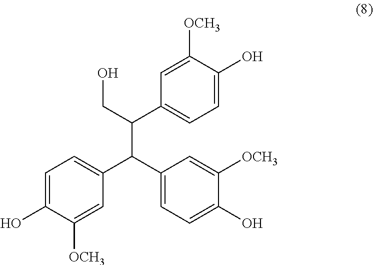 [SMILES: COc1cc(C(CO)C(c2ccc(O)c(OC)c2)c2ccc(O)c(OC)c2)ccc1O]
said process comprising the step of:
i. deprotecting a compound of formula (7) 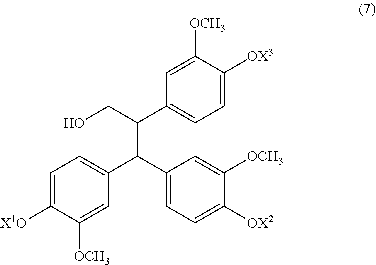
to obtain said compound of formula (8),
wherein X′, X², and X³ is a suitable protecting group for a hydroxyl group.

39. The process of claim 38, wherein deprotecting is with ammonium formate (HCO₂NH₄) and palladium on carbon (Pd/C).

40. The process of claim 38, wherein deprotecting is with ammonium formate (HCO₂NH₄) and palladium on carbon (Pd/C) in methanol (MeOH).

41. A process for the synthesis of a compound of formula (8) (Quebecol) 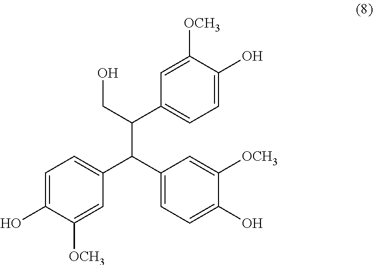 [SMILES: COc1cc(C(CO)C(c2ccc(O)c(OC)c2)c2ccc(O)c(OC)c2)ccc1O]
said process comprising the step of:
i. reacting a compound of formula (1) 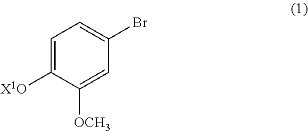
with a compound of formula (2) 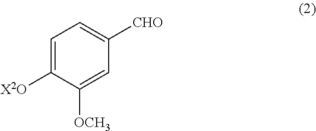
in presence of a strong base, to obtain a compound of formula (3) 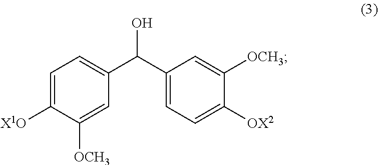
ii. brominating said compound of formula (3), to obtain a compound of formula (4) 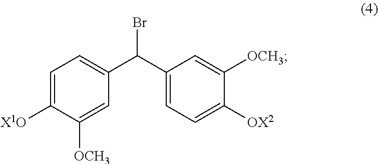
iii. reacting said compound of formula (4) with a compound of formula (5) 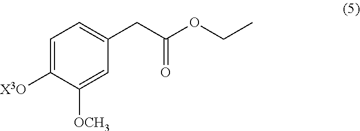
in the presence of a strong base, to obtain a compound of formula (6);
iv. reducing said compound of formula (6) to obtain a compound of formula (7) 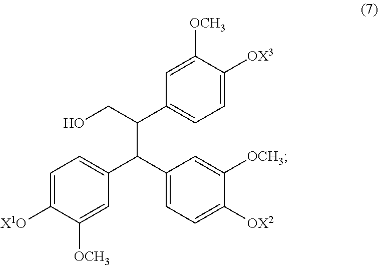
v. deprotecting said compound of formula (7) to obtain said compound of formula (8) (Quebecol),
wherein X¹, X², and X³ is a suitable protecting group for a hydroxyl group.

42. The process of any one of claims 14 to 41, wherein said suitable protecting group for a hydroxyl group is chosen from C₁-C₂₅ ethers, C₁-C₂₅ substituted methyl ethers, C₁-C₂₅ substituted ethyl ethers, C₁-C₂₅ acyl groups, C₁-C₂₅ halogenated acyl groups, C₁-C₂₅ substituted benzyl ethers, C₁-C₂₅ silyl ethers, C₁-C₂₅ esters, C₁-C₂₅ carbonates, and C₁-C₂₅ sulfonates.

43. The process of any one of claims 14 to 42, wherein said suitable protecting group for a hydroxyl group is chosen from diphenylmethylchlorosilane (DPMS), Tosyl, methyl, methoxymethyl, benzyloxymethyl, tetrahydropyranyl, tetrahydrofuranyl, 2-(trimethylsilyl)ethoxymethyl, dioxanyl, 1-ethoxyethyl, 1-(2-chloroethoxy)ethyl, 2,2,2-trichloroethyl, t-butyl, allyl, propargyl, benzyl, p-methoxybenzyl, diphenylmethyl, triphenylmethyl, trimethylsilyl, triethylsilyl, triisopropylsilyl, dimethylisopropylsilyl, diethylisopropylsilyl, dimethylthexylsilyl, t-butyldimethylsilyl, t-butyldiphenylsilyl, tribenzylsilyl, triphenylsilyl, triisopropylsilyl, diphenylmethylsilyl, benzylformate, methylcarbonyl, ethylcarbonyl, methoxymethyl carbonyl, trichloroethoxycarbonyl, benzylcarbonyl, benzyloxycarbonyl. allylsulfonyl, methanesulfonyl, and p-toluenesulfonyl.

44. The process of any one of claims 14 to 42, wherein said suitable protecting group for a hydroxyl group is benzyl (Bn).

45. The process of claim 41, wherein in step i), strong base is n-butyllithium (n-BuLi).

46. The process of claim 45, wherein reacting is in tetrahydrofuran (THF) at −78° C.

47. The process of claim 41, wherein in step ii) brominating is with acetyl bromide (CH₃COBr).

48. The process of claim 41, wherein in step ii) brominating is with acetyl bromide (CH₃COBr) in benzene.

49. The process of claim 41, wherein is step iii) said strong base is Lithium diisopropylamide (LDA).

50. The process of claim 41, wherein in step iii) said strong base is Lithium diisopropylamide (LDA) in tetrahydrofuran at tetrahydrofuran (THF) at −78° C.

51. The process of claim 41, wherein in step iv) reducing is with lithium aluminum hydride (LiAlH₄).

52. The process of claim 41, wherein in step iv) reducing is with lithium aluminum hydride (LiAlH₄) in tetrahydrofuran (THF).

53. The process of claim 41, wherein in step v) deprotecting is with ammonium formate (HCO₂NH₄) and palladium on carbon (Pd/C).

54. The process of claim 41, wherein deprotecting is with ammonium formate (HCO₂NH₄) and palladium on carbon (Pd/C) in methanol (MeOH).

55. A compound of formula (5′) and (9′): 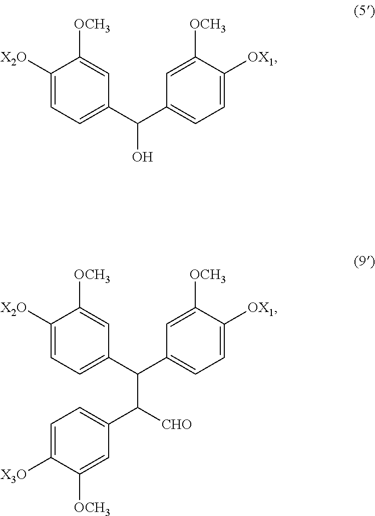
wherein X₁, X₂ and X₃ are as defined above.

56. A compound of formula (3), (4), (6) and (7): 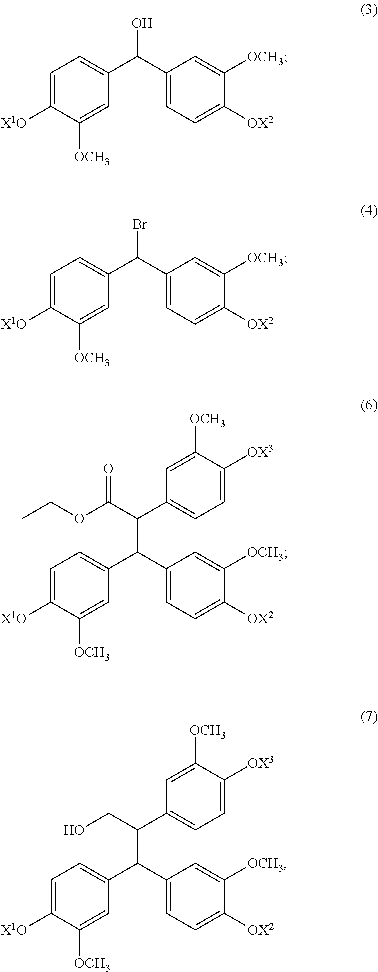
wherein X¹, X² and X³ areas defined above.
